(19) United States
(12) Patent Application Publication
(10) Pub. No.: US 2017/0137355 A1
(43) Pub. Date: May 18, 2017

(54) Carbon Efficient Process for Converting Methane to Olefins and Methanol by Oxidative Coupling of Methane

(71) Applicant: Sabic Global Technologies, B.V., BERGEN OP ZOOM (NL)

(72) Inventors: Vidya Sagar Reddy SARSANI, Pearland, TX (US); Aghaddin MAMEDOV, Sugar Land, TX (US); David WEST, Bellaire, TX (US); Wugeng LIANG, Richmond, TX (US)

(21) Appl. No.: 15/332,631

(22) Filed: Oct. 24, 2016

Related U.S. Application Data
(60) Provisional application No. 62/255,675, filed on Nov. 16, 2015.

(51) Int. Cl. C07C 29/48; C07C 2/82; C07C 5/32; C01B 3/36

(52) U.S. Cl. CPC C07C 29/48; C01B 3/36; C07C 2/82; C07C 5/32; C01B 2203/0255; C01B 2203/061; C01B 2203/062; C01B 2203/1241

(57) ABSTRACT

A method for producing olefins and methanol comprising introducing a first reactant mixture comprising CH₄ and O₂ to a first reaction zone; allowing the first reactant mixture to react via an OCM reaction to form a first product mixture characterized by a first H₂/CO molar ratio; introducing a second reactant mixture comprising the first product mixture and an ethane stream to a second reaction zone, wherein ethane of the second reactant mixture undergoes a cracking reaction to produce ethylene; recovering a second product mixture from the second reaction zone, wherein the second product mixture is characterized by a second H₂/CO molar ratio, and wherein the second H₂/CO molar ratio is greater than the first H₂/CO molar ratio; recovering from the second product mixture a methanol production feed stream comprising methane, H₂ and CO; and introducing the methanol production feed stream to a third reaction zone to produce methanol.

### CROSS-REFERENCE TO RELATED APPLICATIONS

The present application is a non-provisional of and claims priority to U.S. Provisional Patent Application No. 62/255,675 filed Nov. 16, 2015 and entitled “Carbon Efficient Process for Converting Methane to Olefins and Methanol by Oxidative Coupling of Methane,” which application is incorporated by reference herein in its entirety.

### TECHNICAL FIELD

The present disclosure relates to methods of producing hydrocarbons and alcohols, more specifically methods of producing olefins and methanol by oxidative coupling of methane.

### BACKGROUND

Hydrocarbons, and specifically olefins such as ethylene, can be typically used to produce a wide range of products, for example, break-resistant containers and packaging materials. Currently, for industrial scale applications, ethylene is produced by heating natural gas condensates and petroleum distillates, which include ethane and higher hydrocarbons, and the produced ethylene is separated from a product mixture by using gas separation processes.

Ethylene can also be produced by oxidative coupling of the methane (OCM) as represented by Equations (I) and (II):

2CH₄+O₂→C₂H₄+2H₂O ΔH=−67 kcal/mol (I)

2CH₄+1/2O₂→C₂H₆+H₂O ΔH=−42 kcal/mol (II)

Oxidative conversion of methane to ethylene is exothermic. Excess heat produced from these reactions (Equations (I) and (II)) can push conversion of methane to carbon monoxide and carbon dioxide rather than the desired C₂ hydrocarbon product (e.g., ethylene):

CH₄+1.5O₂→CO+2H₂OΔH=−124 kcal/mol (III)

CH₄+2O₂→CO₂+2H₂OΔH=−192 kcal/mol (IV)

The excess heat from the reactions in Equations (III) and (IV) further exasperate this situation, thereby substantially reducing the selectivity of ethylene production when compared with carbon monoxide and carbon dioxide production.

Additionally, while the overall OCM is exothermic, catalysts are used to overcome the endothermic nature of the C—H bond breakage. The endothermic nature of the bond breakage is due to the chemical stability of methane, which is a chemically stable molecule due to the presence of its four strong tetrahedral C—H bonds (435 kJ/mol). When catalysts are used in the OCM, the exothermic reaction can lead to a large increase in catalyst bed temperature and uncontrolled heat excursions that can lead to catalyst deactivation and a further decrease in ethylene selectivity. Furthermore, the produced ethylene is highly reactive and can form unwanted and thermodynamically favored oxidation products.

There have been attempts to control the exothermic reaction of the OCM by using alternating layers of selective OCM catalysts; through the use of fluidized bed reactors; and/or by using steam as a diluent. However, these solutions are costly and inefficient. For example, a large amount of water (steam) is required to absorb the heat of the reaction.

Methanol can also be used to produce a wide range of products, such as of paints, solvents and plastics, and has found innovative applications in energy, transportation fuel and fuel cells. Methanol is commonly produced from synthesis gas. However, the formation of synthesis gas is strongly endothermic and requires high temperatures, which translates in a high energy input. Thus, there is an ongoing need for the development of processes for the production of olefins such as ethylene, and methanol.

### BRIEF SUMMARY

A method for producing olefins and methanol comprising (a) introducing a first reactant mixture to a first reaction zone, wherein the first reactant mixture comprises methane (CH₄) and oxygen (O₂), and wherein the first reaction zone is characterized by a first reaction zone temperature of from about 700° C. to about 1,100° C., (b) allowing at least a portion of the first reactant mixture to react via an oxidative coupling of CH₄ (OCM) reaction to form a first product mixture, wherein the first product mixture comprises C₂₊ hydrocarbons, hydrogen (H₂), carbon monoxide (CO), water, CO₂, and unreacted methane, wherein the first product mixture is characterized by a first hydrogen (H₂) to carbon monoxide (CO) (H₂/CO) molar ratio, wherein the C₂₊ hydrocarbons comprise C₂ hydrocarbons and C₃₊ hydrocarbons, and wherein the C₂ hydrocarbons comprise ethane (C₂H₆) and ethylene (C₂H₄), (c) introducing a second reactant mixture comprising at least a portion of the first product mixture and an ethane stream to a second reaction zone, wherein the second reaction zone is characterized by a second reaction zone temperature of from about 750° C. to about 1,000° C., and wherein at least a portion of ethane of the second reactant mixture undergoes a cracking reaction to produce ethylene, (d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio, and wherein the second H₂/CO molar ratio is greater than the first H₂/CO molar ratio, (e) recovering a methanol production feed stream from at least a portion of the second product mixture, wherein the methanol production feed stream comprises methane, H₂ and CO, and (f) introducing at least a portion of the methanol production feed stream to a third reaction zone comprising a catalyst to produce a methanol stream and a methane-rich stream, wherein at least a portion of the methane-rich stream is recycled to the first reaction zone.

A method for producing ethylene and methanol comprising (a) introducing a first reactant mixture to a first reaction zone, wherein the first reactant mixture comprises methane (CH₄) and oxygen (O₂), wherein the first reaction zone is characterized by a first reaction zone temperature of from about 800° C. to about 1,000° C., wherein the first reaction zone is characterized by a residence time of from about 250 milliseconds to about 750 milliseconds, and wherein the first reaction zone excludes a catalyst, (b) allowing at least a portion of the first reactant mixture in the first reaction zone to react via an oxidative coupling of CH₄ reaction to form a first product mixture, wherein the first product mixture comprises C₂₊ hydrocarbons, hydrogen (H₂), carbon monoxide (CO), water, CO₂, and unreacted methane, wherein the first product mixture is characterized by a first hydrogen (H₂) to carbon monoxide (CO) (H₂/CO) molar ratio of from about 0.5:1 to about 2.0:1, wherein the C₂₊ hydrocarbons comprise C₂ hydrocarbons and C₃₊ hydrocarbons, and wherein the C₂ hydrocarbons comprise ethane and ethylene, (c) introducing a second reactant mixture comprising at least a portion of the first product mixture and an ethane stream to a second reaction zone, wherein the second reactant mixture is characterized by a C₂H₆/CH₄ molar ratio of from about 0.01:1 to about 0.5:1, wherein the second reaction zone is characterized by a second reaction zone temperature of from about 800° C. to about 1,000° C., wherein the second reaction zone is characterized by a residence time of from about 200 milliseconds to about 800 milliseconds, and wherein at least a portion of ethane of the second reactant mixture undergoes a cracking reaction to produce ethylene, (d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio of from about 0.8:1 to about 2.5:1, and wherein the second H₂/CO molar ratio is greater than the first H₂/CO molar ratio, (e) recovering a methanol production feed stream from at least a portion of the second product mixture, wherein the methanol production feed stream comprises methane, H₂ and CO, and (f) introducing at least a portion of the methanol production feed stream to a third reaction zone comprising a Cu/Zn/Al₂O₃ catalyst to produce a methanol stream and a methane-rich stream, wherein at least a portion of the methane-rich stream is recycled to the first reaction zone.

A method for producing olefins and methanol comprising (a) introducing a first reactant mixture to a first reaction zone, wherein the first reactant mixture comprises methane (CH₄) and oxygen (O₂), and wherein the first reaction zone is characterized by a first reaction zone temperature of from about 700° C. to about 1,100° C., (b) allowing at least a portion of the first reactant mixture to react via an oxidative coupling of CH₄ reaction to form a first product mixture, wherein the first product mixture comprises C₂₊ hydrocarbons, hydrogen (H₂), carbon monoxide (CO), water, CO₂, and unreacted methane, wherein the first product mixture is characterized by a first hydrogen (H₂) to carbon monoxide (CO) (H₂/CO) molar ratio, wherein the C₂₊ hydrocarbons comprise C₂ hydrocarbons and C₃₊ hydrocarbons, and wherein the C₂ hydrocarbons comprise ethane (C₂H₆) and ethylene (C₂H₄), (c) introducing a second reactant mixture comprising at least a portion of the first product mixture and an ethane stream to a second reaction zone, wherein the second reaction zone is characterized by a second reaction zone temperature of from about 750° C. to about 1,000° C., and wherein at least a portion of ethane of the second reactant mixture undergoes a cracking reaction to produce ethylene, (d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio, and wherein the second H₂/CO molar ratio is greater than the first H₂/CO molar ratio, (e) recovering ethylene from at least a portion of the second product mixture, and (f) recovering at least a portion of the H₂ and at least a portion of the CO from the second product mixture to yield a recovered synthesis gas.

A method for producing olefins and methanol comprising (a) reacting, in the absence of a catalyst, methane (CH₄) and oxygen (O₂) via an oxidative coupling reaction in a first reaction zone to form a first product mixture, wherein the first product mixture comprises ethane, ethylene, carbon monoxide (CO), hydrogen (H₂), water, carbon dioxide (CO₂), and unreacted methane, (b) introducing at least a portion of the first product mixture to a second reaction zone, wherein at least a portion of the ethane undergoes a [steam] cracking reaction to produce ethylene, (c) recovering a second product mixture from the second reaction zone, wherein the second product mixture comprises ethane, ethylene, carbon monoxide (CO), hydrogen (H₂), water, CO₂, and unreacted methane, and (d) reacting at least a portion of the unreacted methane, CO, and H₂ from the second product mixture in a third reaction zone to form methanol.

A method for producing olefins and methanol comprising (a) introducing a first reactant mixture to a first reaction zone, wherein the first reactant mixture comprises methane (CH₄) and oxygen (O₂), wherein the first reaction zone is characterized by a first reaction zone temperature of from about 800° C. to about 1,000° C., wherein the first reaction zone is characterized by a residence time in a catalyst bed of from about 20 milliseconds to about 50 milliseconds, and wherein the first reaction zone is catalyzed by an OCM catalyst comprising 2% MnO-5% Na₂WO₄/SiO₂, (b) allowing at least a portion of the first reactant mixture in the first reaction zone to react via an oxidative coupling of CH₄ reaction to form a first product mixture, wherein the first product mixture comprises C₂₊ hydrocarbons, hydrogen (H₂), carbon monoxide (CO), water, carbon dioxide (CO₂), and unreacted methane, wherein the first product mixture is characterized by a first H₂/CO molar ratio of from about 0.5:1 to about 1:1, wherein the C₂₊ hydrocarbons comprise C₂ hydrocarbons and C₃₊ hydrocarbons, and wherein the C₂ hydrocarbons comprise ethane and ethylene, (c) introducing a second reactant mixture comprising at least a portion of the first product mixture and an ethane stream to a second reaction zone, wherein the second reactant mixture is characterized by a C₂H₆/CH₄ molar ratio of from about 0.01:1 to about 0.5:1, wherein the second reaction zone is characterized by a second reaction zone temperature of from about 800° C. to about 1,000° C., wherein the second reaction zone is characterized by a residence time of from about 200 milliseconds to about 800 milliseconds, and wherein at least a portion of ethane of the second reactant mixture undergoes a cracking reaction to produce ethylene, (d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio of from about 0.8:1 to about 2.5:1, and wherein the second H₂/CO molar ratio is greater than the first H₂/CO molar ratio, (e) recovering a methanol production feed stream from at least a portion of the second product mixture, wherein the methanol production feed stream comprises methane, H₂ and CO, and (f) introducing at least a portion of the methanol production feed stream to a third reaction zone comprising a Cu/Zn/Al₂O₃ catalyst to produce a methanol stream and a methane-rich stream, wherein at least a portion of the methane-rich stream is recycled to the first reaction zone.

### BRIEF DESCRIPTION OF THE DRAWINGS

For a detailed description of the preferred embodiments of the disclosed methods, reference will now be made to the accompanying drawing in which:

FIG. 1 displays a schematic of a process that integrates oxidative coupling of methane with methanol production.

### DETAILED DESCRIPTION

Disclosed herein are methods for producing olefins and methanol comprising (a) introducing a first reactant mixture to a first reaction zone, wherein the first reactant mixture comprises methane (CH₄) and oxygen (O₂), and wherein the first reaction zone is characterized by a first reaction zone temperature of from about 700° C. to about 1,100° C.; (b) allowing at least a portion of the first reactant mixture to react via an oxidative coupling of CH₄ (OCM) reaction to form a first product mixture, wherein the first product mixture comprises C₂₊ hydrocarbons, hydrogen (H₂), carbon monoxide (CO), water, CO₂, and unreacted methane, wherein the first product mixture is characterized by a first H₂ to CO (H₂/CO) molar ratio, wherein the C₂₊ hydrocarbons comprise C₂ hydrocarbons and C₃₊ hydrocarbons, and wherein the C₂ hydrocarbons comprise ethane (C₂H₆) and ethylene (C₂H₄); (c) introducing a second reactant mixture comprising at least a portion of the first product mixture and an ethane stream to a second reaction zone, wherein the second reaction zone is characterized by a second reaction zone temperature of from about 750° C. to about 1,000° C., and wherein at least a portion of ethane of the second reactant mixture undergoes a cracking reaction to produce ethylene; (d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio, and wherein the second H₂/CO molar ratio is greater than the first H₂/CO molar ratio; (e) recovering a methanol production feed stream from at least a portion of the second product mixture, wherein the methanol production feed stream comprises methane, H₂ and CO; and (f) introducing at least a portion of the methanol production feed stream to a third reaction zone comprising a catalyst to produce a methanol stream and a methane-rich stream, wherein at least a portion of the methane-rich stream is recycled to the first reaction zone. In an embodiment, the first reaction zone can exclude a catalyst (e.g., a catalyst that catalyzes an OCM reaction). In an embodiment, the second reaction zone can exclude a catalyst (e.g., a catalyst that catalyzes an ethane cracking reaction).

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an embodiment,” “another embodiment,” “other embodiments,” “some embodiments,” and so forth, means that a particular element (e.g., feature, structure, property, and/or characteristic) described in connection with the embodiment is included in at least an embodiment described herein, and may or may not be present in other embodiments. In addition, it is to be understood that the described element(s) can be combined in any suitable manner in the various embodiments.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through the carbon of the carbonyl group.

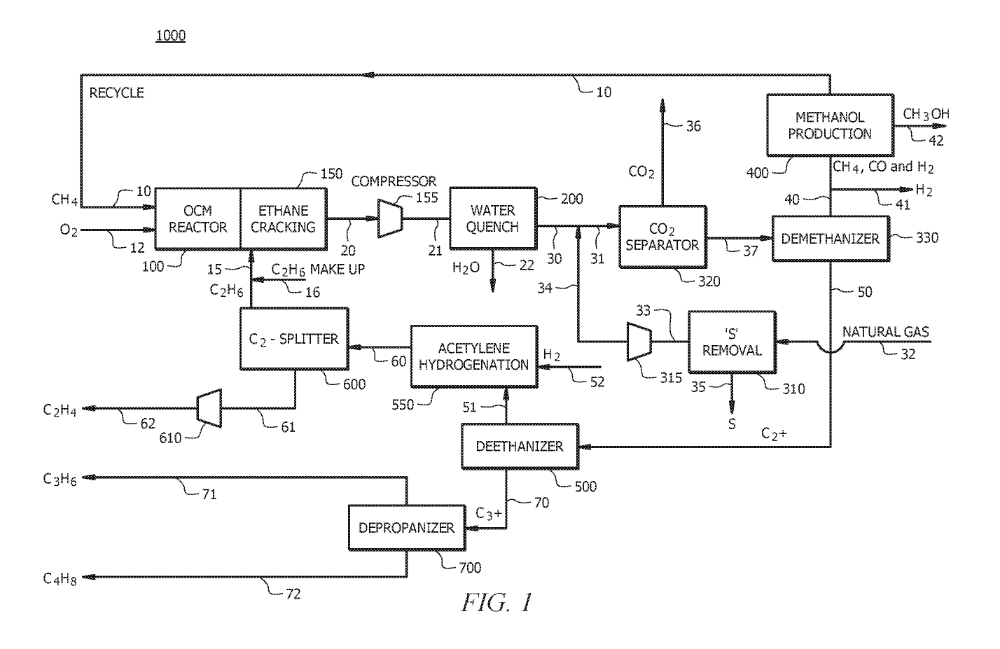

Referring to the embodiment of FIG. 1, an olefin and methanol production system 1000 is disclosed. The olefin and methanol production system 1000 generally comprises an oxidative coupling of CH₄ (OCM) reactor 100; an ethane cracking reactor 150; a water quench vessel 200; a desulphurization vessel 310; a carbon dioxide (CO₂) separator 320; a demethanizer column 330; a methanol production reactor 400; a deethanizer column 500; an acetylene hydrogenation reactor 550; a C₂ splitter column 600; and a depropanizer column 700. In some embodiments, the OCM reactor 100 and the ethane cracking reactor 150 can be the same common reactor. In other embodiments, the OCM reactor 100 and the ethane cracking reactor 150 can be different (e.g., distinct, separate, etc.) reactors (e.g., a first reactor, and a second reactor, respectively). As will be appreciated by one of skill in the art, and with the help of this disclosure, olefin and methanol production system components can be in fluid communication with each other through any suitable conduits (e.g., pipes, streams, etc.).

In an embodiment, a method for producing olefins and methanol can comprise introducing a first reactant mixture to a first reaction zone, wherein the first reactant mixture comprises methane (CH₄) and oxygen (O₂); and allowing at least a portion of the first reactant mixture to react via an OCM reaction to form a first product mixture. In some embodiments, the first reaction zone may exclude an OCM catalyst (e.g., a catalyst that catalyzes an OCM reaction). In other embodiments, the first reaction zone may comprise an OCM catalyst (e.g., a catalyst that catalyzes an OCM reaction).

In an embodiment, a methane-rich stream 10 can be recycled to the OCM reactor 100 from the methanol production reactor 400, as will be disclosed in more detail later herein. In such embodiment, the methane-rich stream 10 can provide the CH₄ for the first reactant mixture. In an embodiment, the methane-rich stream 10 can further comprise H₂. In an embodiment, an O₂ stream 12 can be communicated to the OCM reactor 100, wherein the O₂ stream 12 can provide for the O₂ of the first reactant mixture.

Referring to the embodiment of FIG. 1, when the process is run for the first time, a natural gas stream 32 can be introduced into the methanol production system 1000. Such natural gas stream 32 can be run through purification steps, as will be disclosed in more detail later herein, and then through the methanol production reactor 400 to the recycle stream 10.

The OCM has been the target of intense scientific and commercial interest for more than thirty years due to the tremendous potential of such technology to reduce costs, energy, and environmental emissions in the production of ethylene (C₂H₄). As an overall reaction, in the OCM, CH₄ and O₂ react exothermically to form C₂H₄, water (H₂O) and heat.

Oxidation of methane at high temperatures (e.g., from about 700° C. to about 1,100° C.) can lead to the appearance of the following reactions, as shown in reactions (1)-(4):

2CH₄+O₂=C₂H₄=2H₂O, ΔH=−67 kcal/mol (1)

2CH₄+1/2O₂=C₂H₆+H₂O, ΔH=−42 kcal/mol (2)

CH₄+1.5O₂=CO+2H₂O, ΔH=−124 kcal/mol (3)

CH₄+2O₂=CO₂+2H₂O, ΔH=−192 kcal/mol (4)

Generally, when the OCM is conducted in the absence of an OCM catalyst, conversion of methane is low and the main products of conversion are CO and CO₂, as thermodynamically favored by reactions (3) and (4).

In some embodiments, a method for producing olefins and methanol as disclosed herein can comprise conducting an OCM reaction in the absence of an OCM catalyst, by controlling a range of first reaction zone temperature, a first reaction zone residence time and a first reaction zone feed composition (e.g., a first reactant mixture composition) in such a way to maximize a C₂₊ selectivity and the production of a high H₂/CO molar ratio (e.g., from about 0.3:1 to about 2:1), thereby minimizing CO₂ formation by reaction (4), as will be described in more detail later herein. In some embodiments, controlling a first reaction zone feed composition can further comprise introducing to the reactor (e.g., OCM reactor 100, non-catalytic OCM reactor) other components (e.g., reagents other than methane and oxygen), such as for example hydrogen, thereby changing the pathway of methane conversion reactions, as will be described in more detail later herein. In an embodiment, non-catalytic OCM processes and reactors are described in more detail in U.S. Provisional Application No. 62/183,456 [4515-00800/14T&I0082], which is incorporated by reference herein in its entirety, and which is appended hereto.

Generally, in a catalytic OCM process, CH₄ is first oxidatively converted into ethane (C₂H₆), and then into C₂H₄. CH₄ is activated heterogeneously on a catalyst surface, forming methyl free radicals (e.g., CH₃.), which then couple in a gas phase to form C₂H₆. C₂H₆ subsequently undergoes dehydrogenation to form C₂H₄. An overall yield of desired C₂ hydrocarbons is reduced by non-selective reactions of methyl radicals with the catalyst surface and/or oxygen in the gas phase, which produce (undesirable) carbon monoxide and carbon dioxide. Some of the best reported catalytic OCM outcomes encompass a ˜20% conversion of methane and ˜80% selectivity to desired C₂ hydrocarbons.

In other embodiments, a method for producing olefins and methanol as disclosed herein can comprise conducting an OCM reaction in the presence of an OCM catalyst. In such embodiments, the OCM catalyst can comprise basic oxides; mixtures of basic oxides; redox elements; redox elements with basic properties; mixtures of redox elements with basic properties; mixtures of redox elements with basic properties promoted with alkali and/or alkaline earth metals; rare earth metal oxides; mixtures of rare earth metal oxides; mixtures of rare earth metal oxides promoted by alkali and/or alkaline earth metals; manganese; manganese compounds; lanthanum; lanthanum compounds; sodium; sodium compounds; cesium; cesium compounds; calcium; calcium compounds; and the like; or combinations thereof.

Nonlimiting examples of OCM catalysts suitable for use in the present disclosure include CaO, MgO, BaO, CaO—MgO, CaO—BaO, Li/MgO, MnO₂, W₂O₃, SnO₂, MnO₂—W₂O₃, MnO₂—W₂O₃—Na₂O, MnO₂—W₂O₃—Li₂O, La₂O₃, SrO/La₂O₃, CeO₂, Ce₂O₃, La/MgO, La₂O₃—CeO₂, La₂O₃—CeO₂—Na₂O, La₂O₃—CeO₂—CaO, Na—Mn—La₂O₃/Al₂O₃, Na—Mn—O/SiO₂, Na₂WO₄—Mn/SiO₂, Na₂WO₄—Mn—O/SiO₂, and the like, or combinations thereof. In an embodiment, catalytic OCM processes and reactors are described in more detail in U.S. Provisional Application No. 62/209,561 [4515-00600/14T&I0080] and U.S. Provisional Application No. 62/183,453 [4515-00700/14T&I0081], each of which is incorporated by reference herein in its entirety, and each of which is appended hereto.

In an embodiment, the first reactant mixture can comprise a hydrocarbon or mixtures of hydrocarbons, and oxygen. In some embodiments, the hydrocarbon or mixtures of hydrocarbons can comprise natural gas (e.g., CH₄), natural gas liquids, wet natural gas, and the like, or combinations thereof. In an embodiment, the first reactant mixture can comprise CH₄ and O₂.

In an embodiment, the O₂ used in the first reactant mixture can be oxygen gas (which can be obtained via a membrane separation process), technical oxygen (which can contain some air), air, oxygen enriched air, or combinations thereof.

In an embodiment, the first reactant mixture can be a gaseous mixture. In an embodiment, the first reactant mixture can be characterized by a CH₄/O₂ molar ratio of from about 2:1 to about 40:1, alternatively from about 5:1 to about 30:1, alternatively from about 10:1 to about 25:1, alternatively from about 12:1 to about 20:1, or alternatively from about 14:1 to about 18:1.

In an embodiment, the first reactant mixture can further comprise a diluent. The diluent is inert with respect to the OCM reaction, e.g., the diluent does not participate in the OCM reaction. In an embodiment, the diluent can comprise water, nitrogen, inert gases, and the like, or combinations thereof. In an embodiment, the diluent can be present in the first reactant mixture in an amount of from about 0.5% to about 80%, alternatively from about 5% to about 50%, or alternatively from about 10% to about 30%, based on the total volume of the first reactant mixture. In an embodiment, the use of a diluent in an OCM process is described in more detail in U.S. Provisional Application No. 62/209,561 [4515-00600/14T&I0080], which is appended hereto.

In an embodiment, the first reactant mixture can further comprise hydrogen (H₂). Without wishing to be limited by theory, the presence of hydrogen in the first reactant mixture can generate active species (e.g., active radical species), for example by interaction with oxygen, which can further generate new routes for the OCM reaction in the absence of an OCM catalyst. Generally, a stoichiometric equation reaction of hydrogen with oxygen can be described by reaction (5):

2H₂+O₂=2H₂O (5)

Further, without wishing to be limited by theory, at high reaction temperatures (e.g., from about 700° C. to about 1,100° C.), hydrogen and oxygen can create hydroxyl radicals and can propagate an OCM reaction in the presence of methane according to reactions (6)-(9):

H₂+O₂=2OH. (6)

OH.+CH₄=H₂O+CH₃. (7)

CH₃.+O₂=CH₃O₂. (8)

CH₃O₂.=CH₂O+OH. (9)

Without wishing to be limited by theory, hydroxyl radical groups (e.g., OH.) as produced by reaction (6) can abstract hydrogen from methane as shown in reaction (7), which can generate radical active species (e.g., CH₃.) for propagating the OCM reaction similarly to the generation of catalytic active species on a catalyst surface. Reaction (8) can significantly reduce C₂ selectivity. Further, without wishing to be limited by theory, the presence of hydrogen in the first reactant mixture can (i) generate radicals by reaction (6) and (ii) consume oxygen, thereby decreasing the role of reaction (8).

In an embodiment, the first reactant mixture can be characterized by a CH₄/H₂ molar ratio of from about 10:1 to about 100:1, alternatively from about 10:1 to about 50:1, alternatively from about 10:1 to about 20:1, or alternatively from about 8:1 to about 15:1. In an embodiment, a hydrogen molar concentration in the first reactant mixture does not exceed a methane molar concentration in the first reactant mixture.

In an embodiment, the first reactant mixture can be characterized by an O₂/H₂ molar ratio of from about 2:1 to about 10:1, alternatively from about 3:1 to about 9:1, or alternatively from about 5:1 to about 8:1.

In an embodiment, the first reactant mixture can be characterized by a (CH₄+H₂)/O₂ molar ratio of from about 2:1 to about 40:1, alternatively from about 3:1 to about 25:1, alternatively from about 3:1 to about 16:1, alternatively from about 4:1 to about 12:1, or alternatively from about 4:1 to about 8:1.

In an embodiment, the first reactant mixture can be introduced to the first reaction zone at a temperature of from about 150° C. to about 300° C., alternatively from about 175° C. to about 250° C., or alternatively from about 200° C. to about 225° C. As will be appreciated by one of skill in the art, and with the help of this disclosure, while the OCM reaction is exothermic, heat input is necessary for promoting the formation of methyl radicals from CH₄, as the C—H bonds of CH₄ are very stable, and the formation of methyl radicals from CH₄ is endothermic. In an embodiment, the first reactant mixture can be introduced to the reactor at a temperature effective to promote an OCM reaction.

In an embodiment, the first reaction zone can be characterized by a first reaction zone temperature of from about 700° C. to about 1,100° C., alternatively from about 750° C. to about 1,050° C., alternatively from about 800° C. to about 1,025° C., or alternatively from about 950° C. to about 1,000° C.

In an embodiment, the diluent can provide for heat control of the OCM reaction, e.g., the diluent can act as a heat sink. Generally, an inert compound (e.g., a diluent) can absorb some of the heat produced in the exothermic OCM reaction, without degrading or participating in any reaction (OCM or other reaction), thereby providing for controlling a temperature inside the first reaction zone. As will be appreciated by one of skill in the art, and with the help of his disclosure, the diluent can be introduced to the first reaction zone at ambient temperature, or as part of the first reactant mixture (at a first reactant mixture temperature), and as such the temperature of the diluent entering the first reaction zone is much lower than the first reaction zone temperature, and the diluent can act as a heat sink.

In an embodiment, the first reaction zone can be characterized by a pressure of from about ambient pressure (e.g., atmospheric pressure) to about 500 psig, alternatively from about ambient pressure to about 200 psig, or alternatively from about ambient pressure to about 100 psig. In an embodiment, the method for producing olefins by OCM as disclosed herein can be carried out at ambient pressure.

In an embodiment, for non-catalytic OCM, the first reaction zone can be characterized by a residence time of from about 100 milliseconds (ms) to about 30 seconds (s), alternatively from about 150 ms to about 2 s, alternatively from about 300 ms to about 1 s, or alternatively from about 250 ms to about 750 ms. Generally, the residence time of a reaction zone or reactor refers to the average amount of time that a compound (e.g., a molecule of that compound) spends in that particular reaction zone or reactor, e.g., the average amount of time that it takes for a compound (e.g., a molecule of that compound) to travel through the reaction zone or reactor.

In an embodiment, for non-catalytic OCM, the first reaction zone can be characterized by a gas hourly space velocity (GHSV) of from about 30 h⁻¹ to about 20,000 h⁻¹, alternatively from about 1,000 h⁻¹ to about 17,500 h⁻¹, or alternatively from about 5,000 h⁻¹ to about 15,000 h⁻¹. Generally, the GHSV relates a reactant (e.g., reactant mixture, first reactant mixture, second reactant mixture, etc.) gas flow rate to a reaction zone (e.g., first reaction zone, second reaction zone, etc.) or reactor volume. GHSV is usually measured at standard temperature and pressure.

In an embodiment, for catalytic OCM, the first reaction zone can be characterized by a residence time in a catalyst bed of from about 10 milliseconds (ms) to about 200 ms, alternatively from about 20 ms to about 100 ms, or alternatively from about 25 ms to about 75 ms.

In an embodiment, for catalytic OCM, the first reaction zone can be characterized by a gas hourly space velocity (GHSV) of from about 3,600 h⁻¹ to about 36,000 h⁻¹, alternatively from about 5,000 h⁻¹ to about 35,000 h⁻¹, or alternatively from about 10,000 h⁻¹ to about 30,000 h⁻¹.

In some embodiments, a method for producing olefins and methanol can comprise introducing the first reactant mixture to the OCM reactor 100, wherein the OCM reactor can be a non-catalytic OCM reactor. While the non-catalytic OCM reactor excludes a catalyst, the non-catalytic OCM reactor could comprise non-catalytic materials, such as for example ceramic beads, quartz beads, and the like, or combinations thereof.

In an embodiment, the non-catalytic OCM reactor can comprise an isothermal reactor, a fluidized sand bath reactor, an autothermal reactor, an adiabatic reactor, a tubular reactor, a cooled tubular reactor, a continuous flow reactor, a reactor lined with an inert refractory material, a glass lined reactor, a ceramic lined reactor, and the like, or combinations thereof. In an embodiment, the inert refractory material can comprise silica, alumina, silicon carbide, boron nitride, titanium oxide, mullite, mixtures of oxides, and the like, or combinations thereof.

In an embodiment, the isothermal reactor can comprise a tubular reactor, a cooled tubular reactor, a continuous flow reactor, and the like, or combinations thereof.

In an embodiment, the isothermal reactor can comprise a reactor vessel located inside a fluidized sand bath reactor, wherein the fluidized sand bath provides isothermal conditions (i.e., substantially constant temperature) for the reactor. In such embodiment, the fluidized sand bath reactor can be a continuous flow reactor comprising an outer jacket comprising a fluidized sand bath. The fluidized sand bath can exchange heat with the reactor, thereby providing isothermal conditions for the reactor. Generally, a fluidized bath employs fluidization of a mass of finely divided inert particles (e.g., sand particles, metal oxide particles, aluminum oxide particles, metal oxides microspheres, quartz sand microspheres, aluminum oxide microspheres, silicon carbide microspheres) by means of an upward stream of gas, such as for example air, nitrogen, etc.

In an embodiment, the isothermal conditions can be provided by fluidization of heated microspheres around the isothermal reactor, wherein the microspheres can be heated at a temperature of from about 675° C. to about 1,100° C., alternatively from about 700° C. to about 1,050° C., or alternatively from about 750° C. to about 1,000° C.; and wherein the microspheres can comprise sand, metal oxides, quartz sand, aluminum oxide, silicon carbide, and the like, or combinations thereof. In an embodiment, the microspheres (e.g., inert particles) can have a size of from about 10 mesh to about 400 mesh, alternatively from about 30 mesh to about 200 mesh, or alternatively from about 80 mesh to about 100 mesh, based on U.S. Standard Sieve Size.

While in a fluidized state, the individual inert particles become microscopically separated from each other by the upward moving stream of gas. Generally, a fluidized bath behaves remarkably like a liquid, exhibiting characteristics which are generally attributable to a liquid state (e.g., a fluidized bed can be agitated and bubbled; inert particles of less density will float while those with densities greater than the equivalent fluidized bed density will sink; heat transfer characteristics between the fluidized bed and a solid interface can have an efficiency approaching that of an agitated liquid; etc.).

In an embodiment, isothermal conditions can be provided by fluidized aluminum oxide, such as for example by a BFS high temperature furnace, which is a high temperature calibration bath, and which is commercially available from Techne Calibration.

In some embodiments, a method for producing olefins and methanol can comprise introducing the first reactant mixture to the OCM reactor 100, wherein the OCM reactor can be a catalytic OCM reactor.

In such embodiment, the catalytic OCM reactor can comprise an adiabatic reactor, an autothermal reactor, an isothermal reactor, a fluidized sand bath reactor, a tubular reactor, a cooled tubular reactor, a continuous flow reactor, a fixed bed reactor, a fluidized bed reactor, a reactor lined with an inert refractory material, a glass lined reactor, a ceramic lined reactor, and the like, or combinations thereof. In an embodiment, the inert refractory material can comprise silica, alumina, silicon carbide, boron nitride, titanium oxide, mullite, mixtures of oxides, and the like, or combinations thereof. As will be appreciated by one of skill in the art, and with the help of this disclosure, an isothermal reactor of the type described herein can be use either as a non-catalytic OCM reactor or as a catalytic OCM reactor.

In an embodiment, a method for producing olefins and methanol can comprise allowing at least a portion of the first reactant mixture to react via an OCM reaction to form a first product mixture, wherein the first product mixture comprises C₂₊ hydrocarbons, H₂, CO, water, CO₂, and unreacted methane.

In an embodiment, the first product mixture can be characterized by a first H₂/CO molar ratio of from about 0.3:1 to about 2:1, alternatively from about 0.5:1 to about 2:1, or alternatively from about 0.6:1 to about 1.9:1.

In an embodiment, the C₂₊ hydrocarbons can comprise C₂ hydrocarbons and C₃₊ hydrocarbons, wherein the C₂ hydrocarbons can comprise ethane (C₂H₆) and ethylene (C₂H₄). In an embodiment, the C₂ hydrocarbons can further comprise acetylene (C₂H₂). In an embodiment, the C₃₊ hydrocarbons can comprise C₃ hydrocarbons and C₄ hydrocarbons (C₄s), wherein the C₃ hydrocarbons can comprise propane (C₃H₈) and propylene (C₃H₆).

In an embodiment, a method for producing olefins and methanol can comprise introducing a second reactant mixture comprising at least a portion of the first product mixture and an ethane stream to a second reaction zone, wherein at least a portion of ethane of the second reactant mixture undergoes a cracking reaction to produce ethylene. In an embodiment, the second reaction zone excludes a catalyst.

In an embodiment, at least a portion of ethane of the second reactant mixture can undergo a cracking reaction. Generally, a cracking reaction refers to a reaction by which a saturated hydrocarbon or mixture of saturated hydrocarbons is broken down into smaller molecules and/or unsaturated molecules. In the case of ethane cracking, C₂H₆ is converted to C₂H₄ and H₂ according to reaction (10):

C₂H₆=C₂H₄+H₂ (10)

Ethane cracking provides for an increased amount of hydrogen in the second product mixture, which in turn leads to a higher H₂/CO molar ratio, as will be discussed in more detail later herein. Cracking can be done in the presence of steam, and in this case it can be referred to as “steam cracking.” As will be appreciated by one of skill in the art, and with the help of this disclosure, steam for cracking can be supplied by the second reactant mixture that contains at least a portion of the water from the first product mixture. In an embodiment, steam can be optionally introduced to the second reaction zone. As will be appreciated by one of skill in the art, and with the help of this disclosure, if water from the first product mixture is not sufficient for the needs of the steam cracking, additional steam can be introduced into the second reaction zone as necessary.

In an embodiment, at least a portion of the first product mixture can be introduced to the ethane cracking reactor 150.

In some embodiments, a common reactor can comprise both the first reaction zone and the second reaction zone. In such embodiments, the OCM reactor 100 (e.g., non-catalytic OCM reactor, catalytic OCM reactor) and the ethane cracking reactor 150 can be the same common reactor. In such embodiments, the common reactor can comprise a first reaction zone spanning across a first length of the common reactor, and a second reaction zone spanning across a second length of the common reactor, wherein the first length plus the second length can sum up to a total length of the reactor. As will be appreciated by one of skill in the art, and with the help of this disclosure, a first reaction zone and a second reaction zone in a common reactor can be controlled by controlling the residence time in each of the reaction zones of a mixture traveling through the reactor.

In embodiments where a common reactor comprises both the first reaction zone and the second reaction zone, the first reaction zone and the second reaction zone can be characterized by the same temperature and pressure.

In other embodiments, a first reactor (e.g., OCM reactor 100, non-catalytic OCM reactor, catalytic OCM reactor) can comprise the first reaction zone, and a second reactor (e.g., ethane cracking reactor 150) can comprise the second reaction zone. In an embodiment, the first reactor can be characterized by a first pressure and by a first temperature, and the second reactor can be characterized by a second pressure and by a second temperature.

In some embodiments, the first pressure and the second pressure can be the same. In other embodiments, the first pressure and the second pressure can be different.

In some embodiments, the first temperature and the second temperature can be the same. In other embodiments, the first temperature and the second temperature can be different.

In an embodiment, an ethane stream 15 can be communicated from the C₂ splitter column 600 to a second reaction zone. In embodiments where a common reactor comprises both the first reaction zone and the second reaction zone, an ethane stream 15 can be communicated from the C₂ splitter column 600 to the common reactor across a second length of the common reactor, wherein the second length corresponds to the second reaction zone. In embodiments where a first reactor comprises the first reaction zone and a second reactor comprises the second reaction zone, an ethane stream 15 can be communicated from the C₂ splitter column 600 to the second reactor (e.g., ethane cracking reactor 150).

In an embodiment, the ethane stream 15 can be further contacted with additional ethane (e.g., make up C₂H₆ stream 16) prior to step (c) of introducing at least a portion of the first product mixture and the ethane stream to the second reaction zone. In an embodiment, a make up C₂H₆ stream 16 can be communicated to the ethane stream 15. As will be appreciated by one of skill in the art, and with the help of this disclosure, when the C₂ splitter column 600 does not produce enough ethane for the needs of the ethane cracking reactor, the ethane stream 15 can be supplemented as necessary with additional ethane, e.g., make up C₂H₆ stream 16. In an embodiment, a portion of the ethane stream 15 can be recovered and stored for further use. As will be appreciated by one of skill in the art, and with the help of this disclosure, a portion of the ethane stream 15 can be recovered to ensure a desired ratio of ethane to methane in the second reaction zone.

In an embodiment, the second reactant mixture can comprise C₂H₆, C₂H₄, C₃₊ hydrocarbons, H₂, CO, water, CO₂, and methane. In an embodiment, the second reactant mixture can be characterized by a C₂H₆/CH₄ molar ratio of from 0.01 to about 0.5:1, alternatively from about 0.05:1 to about 0.4:1, or alternatively from about 0.1:1 to about 0.2:1.

In an embodiment, the second reaction zone can be characterized by a second reaction zone temperature of from about 750° C. to about 1,000° C., alternatively from about 800° C. to about 1,000° C., or alternatively from about 825° C. to about 950° C.

In an embodiment, the second reaction zone can be characterized by a pressure of from about ambient pressure (e.g., atmospheric pressure) to about 500 psig, alternatively from about ambient pressure to about 200 psig, or alternatively from about ambient pressure to about 100 psig. In an embodiment, the ethane cracking as disclosed herein can be carried out at ambient pressure.

In an embodiment, the second reaction zone can be characterized by a residence time of from about 100 ms to about 2 s, alternatively from about 150 ms to about 1 s, or alternatively from about 200 ms to about 800 ms.

In an embodiment, the second reaction zone can be characterized by a GHSV of from about 30 h⁻¹ to about 20,000 h⁻¹, alternatively from about 1,000 h⁻¹ to about 17,500 h⁻¹, or alternatively from about 5,000 h⁻¹ to about 15,000 h⁻¹.

In an embodiment, a method for producing olefins and methanol can comprise introducing the second reactant mixture to the ethane cracking reactor 150. In such embodiment, the reactor can comprise an isothermal reactor, a fluidized sand bath reactor, an autothermal reactor, an adiabatic reactor, a tubular reactor, a cooled tubular reactor, a continuous flow reactor, a reactor lined with an inert refractory material, a glass lined reactor, a ceramic lined reactor, and the like, or combinations thereof. In an embodiment, the inert refractory material can comprise silica, alumina, silicon carbide, boron nitride, titanium oxide, mullite, mixtures of oxides, and the like, or combinations thereof.

In an embodiment, a method for producing olefins and methanol can comprise recovering a second product mixture from the second reaction zone, wherein the second product mixture comprises C₂₊ hydrocarbons, H₂, CO, water, CO₂, and unreacted methane.

In an embodiment, the second product mixture can be characterized by a second H₂/CO molar ratio, wherein the second H₂/CO molar ratio is greater than the first H₂/CO molar ratio. As will be appreciated by one of skill in the art, and with the help of this disclosure, H₂ is produced in the ethane cracking reaction, and as such the H₂/CO molar ratio increases due to an increased amount of hydrogen.

In an embodiment, the second H₂/CO molar ratio can be controlled by varying a C₂H₆/CH₄ molar ratio of the second reactant mixture. As will be appreciated by one of skill in the art, and with the help of this disclosure, the more ethane is present in the second reactant mixture (e.g., the higher the C₂H₆/CH₄ molar ratio of the second reactant mixture is), the higher the second H₂/CO molar ratio. Further, as will be appreciated by one of skill in the art, and with the help of this disclosure, the C₂H₆/CH₄ molar ratio can be increased as necessary by increasing the ethane in the second reactant mixture. In an embodiment, the C₂H₆/CH₄ molar ratio can be increased by using a make up C₂H₆ stream 16 as depicted in the embodiment of FIG. 1.

In an embodiment, the second H₂/CO molar ratio can be from about 0.5:1 to about 2.5:1, alternatively from about 0.8:1 to about 2.5:1, or alternatively from about 0.6:1 to about 2.0:1.

In an embodiment, a method for producing olefins and methanol can comprise recovering a methanol production feed stream from at least a portion of the second product mixture, wherein the methanol production feed stream comprises methane, H₂ and CO.

In an embodiment, at least a portion of the water can be removed from the second product mixture to yield a dehydrated second product mixture. In an embodiment, a second product mixture stream 20 can be communicated from the ethane cracking reactor to a compressor 155, wherein a compressed second product mixture stream 21 can be communicated from the compressor 155 to a water quench vessel 200. Generally, compressing a gas that contains water from a first pressure to a second pressure (wherein the second pressure is greater than the first pressure) will lead to the water condensing at the second pressure at an increased temperature as compared to a temperature where water of an otherwise similar gas condenses at the first pressure. In an embodiment, the compressed second product mixture stream 21 can be further cooled in the water quench vessel 200 to promote water condensation.

In an embodiment, a water stream 22 can be recovered from the water quench vessel 200. In an embodiment, at least a portion of the water stream 22 can be recycled to a steam stream that can be optionally introduced to the second reaction zone.

In an embodiment, a dehydrated second product mixture stream 30 can be recovered from the water quench vessel 200. In an embodiment, at least a portion of the dehydrated second product mixture stream 30 can be contacted with a purified natural gas stream 34 to yield a methane-rich product mixture stream 31, wherein an amount of methane in the methane-rich product mixture stream 31 is greater than an amount of methane in the dehydrated second product mixture stream 30.

In an embodiment, the purified natural gas stream 34 can be produced by desulphurization of a natural gas stream 32. In an embodiment, a natural gas stream 32 can be communicated to a desulphurization vessel 310. In an embodiment, the natural gas stream 32 can comprise natural gas. In an embodiment, the natural gas stream 32 can further comprise a methane stream from a refinery and/or processing plant. For example, light alkanes, including methane, can often be separated in a refinery during processing of crude oil into various products, and a methane stream can be provided from the same refinery, a different refinery, and/or a refinery off gas. The methane stream can include a stream from combinations of different sources (e.g., streams from different refineries, different streams from the same refinery, etc.). The methane stream can be provided from a remote location and initial processing of the stream (e.g., refining or partial refining) can occur at the remote location to remove certain contaminants; the refining or partial refining can occur on site where the OCM reaction is conducted; or both.

In an embodiment, the natural gas stream 32 can comprise sulfur-containing compounds (e.g., H₂S, SOₓ, such as for example SO₂, S, and/or RS_{y}R′ type compounds). In an embodiment, at least a portion of the sulfur-containing compounds can be removed from the natural gas stream 32 in the desulphurization vessel 310, for example by amine (e.g., monoethanolamine, diethanolamine, etc.) absorption or scavenging. In an embodiment, a sulfur-compound containing stream 35 can be recovered from the desulphurization vessel 310. In an embodiment, a desulphurized natural gas stream 33 can be communicated from the desulphurization vessel 310 to an expander 315. In an embodiment, the purified natural gas stream 34 can be communicated from the expander 315 to the dehydrated second product mixture stream 30 to yield the methane-rich product mixture stream 31. In an embodiment, the expander 315 can bring a pressure of stream 34 to about a pressure of stream 30 prior to mixing stream 34 with stream 30 to yield stream 31. As will be appreciated by one of skill in the art, and with the help of this disclosure, pipeline natural gas can be compressed at high pressures of about 1,000 psi, and as such an expander (e.g., expander 315) would bring the pressure of the natural gas stream to lower values, closer to pressure values of the stream it will be mixed with (e.g., dehydrated second product mixture stream 30).

In an embodiment, at least a portion of CO₂ can be removed from the methane-rich product mixture to yield a purified methane-rich product mixture. In an embodiment, the methane-rich product mixture stream 31 can be communicated to a CO₂ separator 320. In an embodiment, the CO₂ separator 320 can comprise CO₂ removal by amine (e.g., monoethanolamine) absorption (e.g., amine scrubbing), pressure swing adsorption, temperature swing adsorption, gas separation membranes (e.g., porous inorganic membranes, palladium membranes, polymeric membranes, zeolites, etc.), cryogenic separation, and the like, or combinations thereof. In an embodiment, the CO₂ separator 320 can comprise CO₂ removal by amine absorption. In an embodiment, a CO₂ stream 36 can be recovered from the CO₂ separator 320. As will be appreciated by one of skill in the art, and with the help of this disclosure, any residual sulfur-containing compounds entering the CO₂ separator 320 would be removed along with the CO₂, when the separation process is based on amine absorption.

In an embodiment, a purified methane-rich product mixture stream 37 can be communicated from the CO₂ separator 320 to a demethanizer column 330. In an embodiment, the purified methane-rich product mixture stream 37 can exclude sulfur-containing compounds (e.g., SOₓ, such as for example SO₂, S, and/or RS_{y}R′ type compounds). In an embodiment, the purified methane-rich product mixture stream 37 can be substantially free of sulfur-containing compounds, or alternatively essentially free of sulfur-containing compounds. In an embodiment, the purified methane-rich product mixture stream 37 can comprise sulfur-containing compounds in an amount of less than about 1 mol %, alternatively less than about 0.5 mol %, alternatively less than about 0.1 mol %, alternatively less than about 0.01 mol %, alternatively less than about 0.001 mol %, or alternatively less than about 0.0001 mol %.

In an embodiment, the demethanizer column 330 can be a cryogenic distillation column. In an embodiment, the methanol production feed stream 40 and a C₂₊ hydrocarbons stream 50 can be recovered from at least a portion of the purified methane-rich product mixture stream 37 by cryogenic distillation in the demethanizer column 330.

In some embodiments, a portion of H₂ can be recovered as a recovered H₂ stream 41 from the methanol production feed stream 40, for example by pressure swing adsorption. In other embodiments, at least a portion of the methanol production feed stream 40 can be further contacted with additional hydrogen prior to a step (f) of introducing at least a portion of the methanol production feed stream to the third reaction zone. As will be appreciated by one of skill in the art, and with the help of this disclosure, H₂ can either be removed from or added to the methanol production feed stream 40 to maintain a H₂/CO molar ratio optimal for methanol production, which is about 2:1.

In an embodiment, the methanol production feed stream 40 can comprise methane, CO and H₂. In an embodiment, the methanol production feed stream 40 can be characterized by a H₂/CO molar ratio of about 2:1, alternatively about 2.1:1, alternatively from about 1.5:1 to about 2.5:1, alternatively from about 1.8:1 to about 2.3:1, or alternatively from about 2.0:1 to about 2.1:1. In some embodiments, the methanol production feed stream 40 can be characterized by a H₂/CO molar ratio that can be about the same as the second H₂/CO molar ratio.

In an embodiment, a method for producing olefins and methanol can comprise introducing at least a portion of the methanol production feed stream to a third reaction zone comprising a catalyst to produce a methanol stream and a methane-rich stream, wherein at least a portion of the methane-rich stream can be recycled to the first reaction zone. In an embodiment, a methanol production feed stream 40 can be communicated from the demethanizer column 330 to the methanol production reactor 400. In some embodiments, the methanol production feed stream 40 can be pressurized to a pressure of about 1,000 psi prior to introducing stream 40 to the methanol production reactor 400.

In an embodiment, at least a portion of the CO and at least a portion of the H₂ of the methanol production feed stream can undergo a methanol synthesis reaction. Generally, CO and H₂ can be converted into methanol (CH₃OH) according to reaction (11):

CO+2H₂=CH₃OH (11)

Methanol synthesis from CO and H₂ is a catalytic process, and is most often conducted in the presence of copper based catalysts. Nonlimiting examples of catalysts suitable for use in a methanol synthesis reaction from CO and H₂ (e.g., catalysts for the third reaction zone) include Cu, Cu/ZnO, Cu/ThO₂, Cu/Zn/Al₂O₃, Cu/ZnO/Al₂O₃, Cu/Zr, and the like, or combinations thereof.

In an embodiment, at least a portion of methanol production feed stream 40 can be introduced to the methanol production reactor 400 comprising the third reaction zone. The methanol production reactor 400 can comprise any reactor suitable for a methanol synthesis reaction from CO and H₂, such as for example an isothermal reactor, an adiabatic reactor, a slurry reactor, a cooled multi tubular reactor, and the like, or combinations thereof.

In an embodiment, the third reaction zone can be characterized by a third reaction zone temperature of from about 150° C. to about 400° C., alternatively from about 165° C. to about 300° C., or alternatively from about 180° C. to about 250° C.

In an embodiment, the third reaction zone can be characterized by a pressure of from about 750 psig to about 1,500 psig, alternatively from about 1,000 psig to about 1,300 psig, or alternatively from about 1,100 to about 1,200 psig.

In an embodiment, a method for producing olefins and methanol can comprise recovering a CH₃OH stream 42 from the methanol production reactor 400, for example by flashing. In an embodiment, CH₃OH stream 42 comprises CH₃OH, H₂O and heavy alcohols (e.g. C₂₊ alcohols). In an embodiment, a method for producing olefins and methanol can further comprise recovering CH₃OH from the CH₃OH stream 42, for example by distillation.

In an embodiment, a method for producing olefins and methanol can comprise recovering a methane-rich stream 10 from the methanol production reactor 400, for example by distillation. In an embodiment, at least a portion of the methane-rich stream 10 can be communicated to the OCM reactor 100, as previously disclosed herein. In an embodiment, the methane-rich stream 10 can comprise CH₄, CO, CO₂ and H₂.

In an embodiment, the methane-rich stream 10 can comprise CH₄ in an amount of from about 50 mol % to about 99 mol %, alternatively from about 75 mol % to about 95 mol %, or alternatively from about 90 mol % to about 95 mol %. In an embodiment, the methane-rich stream 10 can further comprise H₂ in an amount of from about 0.01 mol % to about 5 mol %, alternatively from about 0.1 mol % to about 2.5 mol %, or alternatively from about 0.5 mol % to about 1 mol %. In an embodiment, the methane-rich stream 10 can further comprise CO in an amount of from about 0.1 mol % to about 10 mol %, alternatively from about 0.25 mol % to about 5 mol %, or alternatively from about 0.5 mol % to about 2 mol %. In an embodiment, the methane-rich stream 10 can further comprise CO₂ in a negligible amount, such as less than about 1 mol %, alternatively less than about 0.1 mol % or alternatively less than about 0.01 mol %.

In an embodiment, a method for producing olefins and methanol can comprise communicating the C₂₊ hydrocarbons stream 50 from the demethanizer column 330 to the deethanizer column 500, wherein the C₂₊ hydrocarbons stream 50 comprises C₂₊ hydrocarbons. In an embodiment, the deethanizer column 500 can be a cryogenic distillation column. In an embodiment, a first C₂ hydrocarbons stream 51 and a C₃₊ hydrocarbons stream 70 can be recovered from at least a portion of C₂₊ hydrocarbons stream 50 by cryogenic distillation, wherein the first C₂ hydrocarbons stream 51 comprises ethylene (C₂H₄), ethane (C₂H₆), and acetylene (C₂H₂), and wherein the C₃₊ hydrocarbons stream 70 comprises C₃ hydrocarbons and C₄ hydrocarbons.

In an embodiment, the first C₂ hydrocarbons stream 51 can comprise C₂H₂ in an amount of from about 0.01 mol % to about 5 mol %, alternatively from about 0.1 mol % to about 4 mol %, or alternatively from about 1.0 mol % to about 2 mol %. As will be appreciated by one of skill in the art, and with the help of this disclosure, C₂H₂ is usually produced as a byproduct in ethane cracking processes, and C₂H₂ can be present in ethane cracking products in amounts of about 1.5 mol %.

In an embodiment, at least a portion of the C₂H₂ in the first C₂ hydrocarbons stream 51 can be contacted with H₂ to yield a second C₂ hydrocarbons stream 60 comprising ethylene, and ethane. In an embodiment, the first C₂ hydrocarbons stream 51 can be communicated from the deethanizer column 500 to the acetylene hydrogenation reactor 550. In an embodiment, a H₂ stream 52 can be communicated to the acetylene hydrogenation reactor 550. In an embodiment, at least a portion of the recovered H₂ stream 41 can be recycled to the H₂ stream 52 used for selective hydrogenation of C₂H₂ in the acetylene hydrogenation reactor 550. The acetylene can be selectively hydrogenated to ethylene by using any suitable methodology such as for example by gas phase hydrogenation.

In an embodiment, an ethylene stream 61 and the ethane stream 15 can be recovered from at least a portion of the second C₂ hydrocarbons stream 60 by cryogenic distillation. In an embodiment, the second C₂ hydrocarbons stream 60 can be communicated from the acetylene hydrogenation reactor 550 to the C₂ splitter column 600. In an embodiment, the C₂ splitter column 600 can be a cryogenic distillation column. In an embodiment, the ethane stream 15 can be communicated from the C₂ splitter column 600 to the ethane cracking reactor 150, as previously disclosed herein. In an embodiment, the ethylene stream 61 can be communicated from the C₂ splitter column 600 to a compressor 610 to yield a pressurized ethylene stream 62. In some embodiments, the pressurized ethylene stream 62 can have a pressure of about 1,000 psi. As will be appreciated by one of skill in the art, and with the help of this disclosure, when ethylene is used as a feedstock for polymerization (e.g., polyethylene production), an ethylene feedstock can have a pressure of about 1,000 psi in some instances.

In an embodiment, a C₃ hydrocarbons stream 71 and a C₄ hydrocarbons stream 72 can be recovered from at least a portion of the C₃₊ hydrocarbons stream 70, wherein the C₃ hydrocarbons stream 71 comprise propylene (C₃H₆), and propane (C₃H₈). In an embodiment, the C₃₊ hydrocarbons stream 70 can be communicated from the deethanizer column 500 to the depropanizer column 700. In an embodiment, the depropanizer column 700 can be a cryogenic distillation column. In an embodiment, the C₃ hydrocarbons stream 71 and the C₄ hydrocarbons stream 72 can be recovered from the depropanizer column 700.

Generally, a selectivity to a desired product or products refers to how much desired product was formed divided by the total products formed, both desired and undesired. For purposes of the disclosure herein, the selectivity to a desired product is a % selectivity based on moles converted into the desired product. Further, for purposes of the disclosure herein, a Cₓ selectivity (e.g., Cₚₚ selectivity, C₂ selectivity, C₂₊ selectivity, etc.) can be calculated by dividing a number of moles of carbon (C) from a reactant (e.g., CH₄ for OCM, C₂H₆ for ethane cracking, CO for methanol production) that were converted into a desired product (e.g., C_{C2H4}, C_{CO}, C_{CH3OH}, etc.) by the total number of moles of C from a reactant that were converted into products (e.g., C_{C2H4}, C_{C2H6}, C_{C2H2}, C_{C3H6}, C_{C3H8}, C_{C4s}, C_{CO}, C_{CO2}, C_{CH3OH}, etc.). C_{C2H4}=number of moles of C from reactant (e.g., CH₄ for OCM, C₂H₆ for ethane cracking) that were converted into C₂H₄; C_{C2H6}=number of moles of C from CH₄ that were converted into C₂H₆; C_{C2H2}=number of moles of C from reactant (e.g., CH₄ for OCM, C₂H₆ for ethane cracking) that were converted into C₂H₂; C_{C3H6}=number of moles of C from reactant (e.g., CH₄ for OCM, C₂H₆ for ethane cracking) that were converted into C₃H₆; C_{C3H8}=number of moles of C from reactant (e.g., CH₄ for OCM, C₂H₆ for ethane cracking) that were converted into C₃H₈; C_{C4s}=number of moles of C from reactant (e.g., CH₄ for OCM, C₂H₆ for ethane cracking) that were converted into C₄ hydrocarbons (C₄s); C_{CO2}=number of moles of C from CH₄ that were converted into CO₂; C_{CO}=number of moles of C from CH₄ that were converted into CO; C_{CH3OH}=number of moles of C from CO that were converted into CH₃OH; etc.

In an embodiment, a method for producing olefins and methanol can allow for the formation of coupling products, and partial oxidation products (e.g., partial conversion products, such as CO, H₂, CO₂) during OCM. In an embodiment, the coupling products can comprise olefins (e.g., alkenes, characterized by a general formula CₙH₂ₙ) and paraffins (e.g., alkanes, characterized by a general formula CₙH₂ₙ₊₂).

In an embodiment, a method for producing olefins and methanol can comprise recovering OCM primary products, wherein the OCM primary products comprise C₂₊ hydrocarbons and CO, wherein the C₂₊ hydrocarbons comprise olefins, and wherein a selectivity to OCM primary products (e.g., Cₚₚ OCM selectivity) can be from about 50% to about 95%, alternatively from about 55% to about 90%, or alternatively from about 60% to about 85%. In an embodiment, the primary products can comprise C₂₊ hydrocarbons and CO, wherein the C₂₊ hydrocarbons can comprise C₂ hydrocarbons, C₃ hydrocarbons, and C₄ hydrocarbons (C₄s), such as for example butane, iso-butane, n-butane, butylene, etc.

The Cₚₚ OCM selectivity refers to how much primary products (e.g., desired products, such as C₂ hydrocarbons, C₃ hydrocarbons, C₄s, CO, etc.) were produced from the OCM divided by the total products produced by OCM, including C₂H₄, C₂H₂, C₃H₆, C₂H₆, C₃H₈, C₄s, CO and CO₂. For example, the Cₚₚ OCM selectivity can be calculated by using equation (12):

$${C}_{pp} OCMselectivity = \frac{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{CO}\end{matrix}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{{CO}_{2}} + {C}_{CO}\end{matrix}} \times 100 \% \qquad ( 12 )$$

As will be appreciated by one of skill in the art, if a specific product and/or hydrocarbon product is not recovered from a certain process (e.g., OCM, ethane cracking, methanol production), then the corresponding C_{Cx} is 0, and the term is simply removed from selectivity calculations.

In an embodiment, a first selectivity to ethylene (C₂₌ first selectivity) can be from about 22% to about 70%, alternatively from about 27% to about 60%, or alternatively from about 32% to about 50%. The C₂₌ first selectivity refers to how much C₂H₄ was produced by the OCM, divided by the total products produced in the OCM, including C₂H₄, C₂H₂, C₃H₆, C₂H₆, C₃H₈, C₄s, CO and CO₂. For example, the first selectivity to ethylene can be calculated by using equation (13):

$${C}_{2} = first selectivity = \frac{2 {C}_{{C}_{2} {H}_{4}}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{{CO}_{2}} + {C}_{CO}\end{matrix}} \times 100 \% \qquad ( 13 )$$

In an embodiment, a second selectivity to ethylene (C₂₌ second selectivity) can be from about 60% to about 98%, alternatively from about 75% to about 95%, or alternatively from about 80% to about 90%. The C₂₌ second selectivity refers to how much C₂H₄ was produced by ethane cracking, divided by the total products produced in ethane cracking, including CH₄, C₂H₄, C₃H₆, C₂H₂, C₃H₈, and C₄s. For example, the second selectivity to ethylene can be calculated by using equation (14):

$${C}_{2} = second selectivity = \frac{2 {C}_{{C}_{2} {H}_{4}}}{{C}_{{CH}_{4}} + 2 {C}_{{C}_{2} {H}_{4}} + 2 {C}_{{C}_{2} {H}_{2}} + 3 {C}_{{C}_{3} {H}_{6}} + 3 {C}_{{C}_{3} {H}_{8}} + 4 {C}_{{C}_{4} s}} \times 100 \% \qquad ( 14 )$$

In an embodiment, a first selectivity to C₂ hydrocarbons (C₂ first selectivity) can be from about 23% to about 85%, alternatively from about 28% to about 70%, or alternatively from about 33% to about 60%. The C₂ first selectivity refers to how much C₂H₄, C₂H₂, and C₂H₆, were produced by OCM, divided by the total products produced in the OCM, including C₂H₄, C₃H₆, C₂H₂, C₂H₆, C₃H₈, C₄s, CO and CO₂. For example, the C₂ first selectivity can be calculated by using equation (15):

$${C}_{2} first selectivity = \frac{2 {C}_{{C}_{2} {H}_{4}} + 2 {C}_{{C}_{2} {H}_{6}} + 2 {C}_{{C}_{2} {H}_{2}}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{{CO}_{2}} + {C}_{CO}\end{matrix}} \times 100 \% \qquad ( 15 )$$

In an embodiment, a first selectivity to C₂₊ hydrocarbons (C₂₊ first selectivity) can be from about 25% to about 90%, alternatively from about 30% to about 80%, or alternatively from about 35% to about 60%. The C₂₊ first selectivity refers to how much C₂H₄, C₂H₂, C₃H₆, C₂H₆, C₃H₈, and C₄s were produced by OCM, divided by the total products produced in the OCM, including C₂H₄, C₂H₂, C₃H₆, C₂H₆, C₃H₈, C₄s, CO and CO₂. For example, the C₂₊ first selectivity can be calculated by using equation (16):

$${C}_{2 +} first selectivity = \frac{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s}\end{matrix}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{{CO}_{2}} + {C}_{CO}\end{matrix}} \times 100 \% \qquad ( 16 )$$

In an embodiment, a selectivity to methanol (C_{CH3OH} selectivity) can be from about 50% to about 99%, alternatively from about 75% to about 99%, or alternatively from about 95% to about 98%. The C_{CH3OH} selectivity refers to how much CH₃OH was produced in the methanol production reactor, divided by the total products produced in the methanol production reactor, including CH₃OH, and C₂₊ alcohols. For example, the selectivity to methanol can be calculated by using equation (17):

$${C}_{{CH}_{3} OH} selectivity = \frac{{C}_{{CH}_{3} OH}}{{C}_{{CH}_{3} OH} + {C}_{{C}_{2} , alcohols }} \times 100 \% \qquad ( 17 )$$

wherein x depends on the type of heavy alcohols formed.

In an embodiment, a per pass hydrocarbon conversion can be from about 10% to about 50%, alternatively from about 12.5% to about 47.5%, or alternatively from about 15% to about 45%. For purposes of the disclosure herein, the per pass hydrocarbon conversion refers to the hydrocarbon conversion in both the OCM and ethane cracking. Generally, a conversion of a reagent or reactant refers to the percentage (usually mol %) of reagent that reacted to both undesired and desired products, based on the total amount (e.g., moles) of reagent present before any reaction took place. For purposes of the disclosure herein, the conversion of a reagent is a % conversion based on moles converted. For purposes of the disclosure herein, a per pass hydrocarbon conversion refers to how much hydrocarbon (e.g., methane, ethane) was converted into both desired and undesired products during both OCM and ethane cracking divided by how much hydrocarbon was introduced to both the OCM reactor and the ethane cracking reactor. For example, the per pass hydrocarbon conversion can be calculated by using equation (18):

$${Hydrocarbons}_{conversion} = \frac{{Moles}_{{CH}_{4} + {C}_{2} {H}_{6}}^{i n} - {Moles}_{{CH}_{4} + {C}_{2} {H}_{6}}^{out}}{{Moles}_{{CH}_{4} + {C}_{2} {H}_{6}}^{i n}} \times 100 \% \qquad ( 18 )$$

wherein Moles_{CH}_{₄}_{+C}_{₂}_{H}_{₆}ⁱⁿ=number of moles of hydrocarbons (e.g., methane and ethane) that was introduced to the OCM reactor and the ethane cracking reactor; and Moles_{CII}_{₄}_{+C}_{₂}_{II}_{₆}^{out}=number of moles of hydrocarbons (e.g., methane and ethane) that were recovered from the OCM reactor and the ethane cracking reactor.

In an embodiment, an overall hydrocarbon (e.g., methane, ethane) yield can be from about 50% to about 95%, alternatively from about 70% to about 85%, or alternatively from about 75% to about 80%. For purposes of the disclosure herein, the overall hydrocarbon (e.g., methane, ethane) yield refers to the moles of C in all useful recovered products (e.g., C₂ hydrocarbons, C₃ hydrocarbons, C₄s, CH₃OH) divided by the total moles of C that were introduced to the olefin and methanol production system 1000.

In an embodiment, a method for producing olefins and methanol as disclosed herein can further comprise minimizing deep oxidation of methane to CO₂. In an embodiment, the second product mixture can comprise less than about 15 mol % CO₂, alternatively less than about 10 mol % CO₂, or alternatively less than about 5 mol % CO₂.

In an embodiment, equal to or greater than about 5 mol %, alternatively equal to or greater than about 10 mol %, or alternatively equal to or greater than about 15 mol % of the methane in the first reactant mixture can be converted overall to useful recovered products (e.g., C₂ hydrocarbons, C₃ hydrocarbons, C₄s, CH₃OH).

In an embodiment, equal to or greater than about 5 mol %, alternatively equal to or greater than about 10 mol %, or alternatively equal to or greater than about 15 mol % of the methane in the first reactant mixture can be converted overall to methanol.

As will be appreciated by one of sill in the art, and with the help of this disclosure, while the current disclosure is discussed in detail in the context of recovering hydrocarbons and producing methanol from the second product mixture, the second product mixture could be also used for recovering a synthesis gas, and such recovered synthesis gas could be used for any suitable purpose. Synthesis gas, also known as syngas, is generally a gas mixture consisting primarily of CO and H₂, and sometimes CO₂. Synthesis gas can be used for producing olefins; for producing methanol; for producing ammonia and fertilizers; in the steel industry; as a fuel source (e.g., for electricity generation); etc.

In an embodiment, the method for producing olefins and methanol as disclosed herein can comprise recovering ethylene from at least a portion of the second product mixture. In an embodiment, the method for producing olefins and methanol as disclosed herein can further comprise recovering at least a portion of the H₂ and at least a portion of the CO from the second product mixture to yield a recovered synthesis gas. In an embodiment, at least a portion of the recovered synthesis gas can be separated from the second product mixture to yield recovered synthesis gas, for example by cryogenic distillation. As will be appreciated by one of skill in the art, and with the help of this disclosure, the recovery of synthesis gas is generally done as a simultaneous recovery of both H₂ and CO.

In an embodiment, the recovered synthesis gas can be characterized by a H₂/CO molar ratio of about 2:1, wherein at least a portion of the recovered synthesis gas can be used for methanol production as previously disclosed herein.

In an embodiment, the recovered synthesis gas can be characterized by a H₂/CO molar ratio of about 1:1, wherein at least a portion of the recovered synthesis gas can be used for dimethyl ether production.

In an embodiment, the recovered synthesis gas can be characterized by a H₂/CO molar ratio of about 1:1, wherein at least a portion of the recovered synthesis gas can be used for oxo-synthesis of aliphatic aldehydes and/or alcohols. In such embodiment, the alcohol can comprise 2-ethyl hexanol.

In an embodiment, the recovered synthesis gas can be further converted to olefins. For example, the recovered synthesis gas can be converted to alkanes by using a Fisher-Tropsch process, and the alkanes can be further converted by dehydrogenation into olefins.

In an embodiment, the recovered synthesis gas can be further converted to liquid hydrocarbons (e.g., alkanes) by a Fisher-Tropsch process. In such embodiment, the liquid hydrocarbons can be further converted by dehydrogenation into olefins.

In an embodiment, the recovered synthesis gas can be further used as fuel to generate power.

In an embodiment, the recovered synthesis gas can be further converted to methane via a methanation process.

In an embodiment, a method for producing ethylene and methanol can comprise (a) introducing a first reactant mixture to a first reaction zone, wherein the first reactant mixture comprises CH₄ and O₂, wherein the first reaction zone is characterized by a first reaction zone temperature of from about 800° C. to about 1,000° C., wherein the first reaction zone is characterized by a residence time of from about 250 milliseconds to about 750 milliseconds, and wherein the first reaction zone excludes a catalyst; (b) allowing at least a portion of the first reactant mixture in the first reaction zone to react via an oxidative coupling of CH₄ reaction to form a first product mixture, wherein the first product mixture comprises C₂₊ hydrocarbons, H₂, CO, water, CO₂, and unreacted methane, wherein the first product mixture is characterized by a first H₂/CO molar ratio of from about 0.5:1 to about 2:1, wherein the C₂₊ hydrocarbons comprise C₂ hydrocarbons and C₃₊ hydrocarbons, and wherein the C₂ hydrocarbons comprise ethane and ethylene; (c) introducing a second reactant mixture comprising at least a portion of the first product mixture and an ethane stream to a second reaction zone, wherein the second reactant mixture is characterized by a C₂H₆/CH₄ molar ratio of from about 0.01:1 to about 0.5:1, wherein the second reaction zone is characterized by a second reaction zone temperature of from about 800° C. to about 1,000° C., wherein the second reaction zone is characterized by a residence time of from about 200 milliseconds to about 800 milliseconds, and wherein at least a portion of ethane of the second reactant mixture undergoes a cracking reaction to produce ethylene; (d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio of from about 0.8:1 to about 2.5:1, and wherein the second H₂/CO molar ratio is greater than the first H₂/CO molar ratio; (e) recovering a methanol production feed stream from at least a portion of the second product mixture, wherein the methanol production feed stream comprises methane, H₂ and CO; and (f) introducing at least a portion of the methanol production feed stream to a third reaction zone comprising a Cu/Zn/Al₂O₃ catalyst to produce a methanol stream and a methane-rich stream, wherein at least a portion of the methane-rich stream is recycled to the first reaction zone. In such embodiment, a common reactor can comprise both the first reaction zone and the second reaction zone.

In an embodiment, a method for producing ethylene and methanol can comprise (a) reacting, in the absence of a catalyst, CH₄ and O₂ via an oxidative coupling reaction in a first reaction zone to form a first product mixture, wherein the first product mixture comprises ethane, ethylene, CO, H₂, water, CO₂, and unreacted methane; (b) introducing at least a portion of the first product mixture to a second reaction zone, wherein at least a portion of the ethane undergoes a [steam] cracking reaction to produce ethylene; (c) recovering a second product mixture from the second reaction zone, wherein the second product mixture comprises ethane, ethylene, CO, H₂, water, CO₂, and unreacted methane, wherein a H₂/CO molar ratio of the first product mixture is from about 0.5:1 to about 2:1, and wherein a H₂/CO molar ratio of the second product mixture is from about 0.6:1 to about 2:1; (d) recovering ethylene from at least a portion of the second product mixture; (e) reacting at least a portion of the unreacted methane, CO, and H₂ from the second product mixture in a third reaction zone to form methanol; and (f) recovering methanol from the third reaction zone.

In an embodiment, a method for producing ethylene and methanol can comprise (a) introducing a first reactant mixture to a first reaction zone, wherein the first reactant mixture comprises CH₄ and O₂, wherein the first reaction zone is characterized by a first reaction zone temperature of from about 800° C. to about 1,000° C., wherein the first reaction zone is characterized by a residence time in a catalyst bed of from about 20 milliseconds to about 50 milliseconds, and wherein the first reaction zone is catalyzed by an OCM catalyst comprising 2% MnO-5% Na₂WO₄/SiO₂; (b) allowing at least a portion of the first reactant mixture in the first reaction zone to react via an oxidative coupling of CH₄ reaction to form a first product mixture, wherein the first product mixture comprises C₂₊ hydrocarbons, H₂, CO, water, CO₂, and unreacted methane, wherein the first product mixture is characterized by a first H₂/CO molar ratio of from about 0.5:1 to about 1:1, wherein the C₂₊ hydrocarbons comprise C₂ hydrocarbons and C₃₊ hydrocarbons, and wherein the C₂ hydrocarbons comprise ethane and ethylene; (c) introducing a second reactant mixture comprising at least a portion of the first product mixture and an ethane stream to a second reaction zone, wherein the second reactant mixture is characterized by a C₂H₆/CH₄ molar ratio of from about 0.01:1 to about 0.5:1, wherein the second reaction zone is characterized by a second reaction zone temperature of from about 800° C. to about 1,000° C., wherein the second reaction zone is characterized by a residence time of from about 200 milliseconds to about 800 milliseconds, and wherein at least a portion of ethane of the second reactant mixture undergoes a cracking reaction to produce ethylene; (d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio of from about 0.8:1 to about 2.5:1, and wherein the second H₂/CO molar ratio is greater than the first H₂/CO molar ratio; (e) recovering a methanol production feed stream from at least a portion of the second product mixture, wherein the methanol production feed stream comprises methane, H₂ and CO; and (f) introducing at least a portion of the methanol production feed stream to a third reaction zone comprising a Cu/Zn/Al₂O₃ catalyst to produce a methanol stream and a methane-rich stream, wherein at least a portion of the methane-rich stream is recycled to the first reaction zone. In such embodiment, a common reactor can comprise both the first reaction zone and the second reaction zone.

In an embodiment, a method for producing olefins (e.g., ethylene) and methanol as disclosed herein can advantageously display improvements in one or more method characteristics when compared to an otherwise similar method that does not integrate OCM with other processes for producing desired products. Ethane cracking as disclosed herein can advantageously increase both a selectivity to ethylene and produce more hydrogen such that a H₂/CO molar ratio is increased to a value that is closer to that needed for converting H₂ and CO into methanol (e.g., about 2.1:1). A synthesis gas (e.g., H₂ and CO) to methanol conversion process as disclosed herein can increase further the overall efficiency of the process by producing methanol from the H₂ and CO obtained from OCM and ethane cracking.

In an embodiment, a method for producing olefins (e.g., ethylene) and methanol as disclosed herein can advantageously display an increased overall carbon efficiency and/or oxygen utilization (e.g., oxygen conversion) when compared to a carbon efficiency and/or oxygen conversion of a similar OCM process that is not integrated with ethane cracking and synthesis gas to methanol conversion. In such embodiment, the increased overall carbon efficiency of the method can be due to using a new integration scheme of OCM with ethane cracking and methanol production by taking advantage of conversion of large amounts of CO formed in the OCM reaction and hydrogen generated during ethane cracking to additional valuable products such as methanol.

In an embodiment, the method for producing olefins and synthesis gas as disclosed herein can advantageously allow for the use of CO produced in the OCM reaction for the production of methanol, thereby increasing an overall efficiency of the method. While CO is not recovered, a selectivity to CO of the OCM process (C_{CO} selectivity/OCM) can be calculated, by accounting for all the products of the OCM reaction. In embodiments where the first reaction zone excludes an OCM catalyst, the C_{CO} selectivity/OCM can be from about 25% to about 75%, alternatively from about 40% to about 70%, or alternatively from about 50% to about 60%. In embodiments where the first reaction zone comprises an OCM catalyst, the C_{CO} selectivity/OCM can be from about 1% to about 20%, alternatively from about 5% to about 17.5%, or alternatively from about 10% to about 15%. For example, the C_{CO} selectivity/OCM can be calculated by using equation (19):

$${C}_{CO} selectivity / OCM = \frac{{C}_{CO}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 3 {C}_{{C}_{3} {H}_{3}} + 4 {C}_{{C}_{4} s} + {C}_{{CO}_{2}} + {C}_{CO}\end{matrix}} \times 100 \% \qquad ( 19 )$$

wherein all “Cᵢ” of equation (19) refer to number of moles of C from CH₄ that were converted into product “i” during OCM. Additional advantages of the methods for the production of olefins (e.g., ethylene) and methanol as disclosed herein can be apparent to one of skill in the art viewing this disclosure.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Example 1

Oxidative coupling of methane (OCM) reactions were conducted in the absence of a catalyst as follows. Methane and oxygen gases, along with an internal standard, an inert gas (neon) were fed to a quartz reactor (e.g., non-catalytic OCM reactor) with an internal diameter (I.D.) of 4 mm and were heated using a traditional clamshell furnace at a desired set point temperature of 900° C. The reactor was first heated to a desired temperature under an inert gas flow and then a desired gas mixture was fed to the reactor. A CH₄/O₂ feed ratio was 4.

Table 1 below shows methane conversion and selectivity to various products during non-catalytic OCM reaction. Selectivities to products for the OCM reaction were calculated in a manner similar to that outlined in equation (18). Methane conversion for the non-catalytic OCM reactor was calculated by making a ratio between the moles of methane that entered the non-catalytic OCM reactor minus the moles of methane that left the non-catalytic OCM reactor, and the moles of methane that entered the non-catalytic OCM reactor.

TABLE 1

| | | | |
|---|---|---|---|
| Residence time [ms] | 1282 | 656 | 512 |
| % CH4 Conversion | 25.4 | 23.4 | 20.6 |
| % ‘C’ Selectivities | | | |
| C2═ | 21.7 | 26.5 | 28.5 |
| C2 | 3.3 | 5.6 | 8.1 |
| C3═ | 1.4 | 1.8 | 2.1 |
| C3 | 0.3 | 0.4 | 0.5 |
| C2+ | 26.8 | 34.3 | 39.1 |
| CO | 67.7 | 60.9 | 56.5 |
| CO2 | 5.5 | 4.8 | 4.3 |
| H2/CO ratio | 0.30 | 0.35 | 0.66 |

The data in Table 1 show that, for example, at a methane conversion of 20.6%, C₂₊ selectivities of greater than about 39% can be obtained with a CO selectivity of about 56% and a H₂/CO molar ratio of greater than about 0.6. At least a portion of the CO in a product stream could be further converted to methanol, increasing the per pass and overall carbon efficiency before separating methanol and recycling the remaining stream back to an OCM reactor. Addition of ethane to the OCM reactor could increase a H₂/CO molar ratio from about 0.66 (which is the value given in Table 1 for a residence time of 512 ms) to about 1:1, which would render the synthesis gas useful for carbonylation reactions; or even to 2:1, which would render the synthesis gas useful for methanol and/or olefins synthesis.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. §1.72 and the purpose stated in 37 C.F.R. §1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

### ADDITIONAL DISCLOSURE

A first embodiment, which is a method for producing olefins and methanol comprising (a) introducing a first reactant mixture to a first reaction zone, wherein the first reactant mixture comprises methane (CH₄) and oxygen (O₂), and wherein the first reaction zone is characterized by a first reaction zone temperature of from about 700° C. to about 1,100° C.; (b) allowing at least a portion of the first reactant mixture to react via an oxidative coupling of CH₄ (OCM) reaction to form a first product mixture, wherein the first product mixture comprises C₂₊ hydrocarbons, hydrogen (H₂), carbon monoxide (CO), water, CO₂, and unreacted methane, wherein the first product mixture is characterized by a first hydrogen (H₂) to carbon monoxide (CO) (H₂/CO) molar ratio, wherein the C₂₊ hydrocarbons comprise C₂ hydrocarbons and C₃₊ hydrocarbons, and wherein the C₂ hydrocarbons comprise ethane (C₂H₆) and ethylene (C₂H₄); (c) introducing a second reactant mixture comprising at least a portion of the first product mixture and an ethane stream to a second reaction zone, wherein the second reaction zone is characterized by a second reaction zone temperature of from about 750° C. to about 1,000° C., and wherein at least a portion of ethane of the second reactant mixture undergoes a cracking reaction to produce ethylene; (d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio, and wherein the second H₂/CO molar ratio is greater than the first H₂/CO molar ratio; (e) recovering a methanol production feed stream from at least a portion of the second product mixture, wherein the methanol production feed stream comprises methane, H₂ and CO; and (f) introducing at least a portion of the methanol production feed stream to a third reaction zone comprising a catalyst to produce a methanol stream and a methane-rich stream, wherein at least a portion of the methane-rich stream is recycled to the first reaction zone.

A second embodiment, which is the method of the first embodiment, wherein a common reactor comprises both the first reaction zone and the second reaction zone.

A third embodiment, which is the method of the first embodiment, wherein a first reactor comprises the first reaction zone, and wherein a second reactor comprises the second reaction zone.

A fourth embodiment, which is the method of the third embodiment, wherein the first reactor comprises a non-catalytic OCM reactor, and wherein the first reaction zone is characterized by a residence time of from about 100 milliseconds to about 30 seconds.

A fifth embodiment, which is the method of any one of the third and the fourth embodiments, wherein the first reactor comprises a catalytic OCM reactor, and wherein the first reaction zone is characterized by a residence time in a catalyst bed of from about 10 milliseconds to about 200 milliseconds.

A sixth embodiment, which is the method of any one of the first through the fifth embodiments, wherein the second reaction zone is characterized by a residence time of from about 100 milliseconds to about 2 seconds.

A seventh embodiment, which is the method of any one of the first through the sixth embodiments, wherein the first reaction zone and/or the second reaction zone is characterized by a pressure of from about ambient pressure to about 500 psig.

An eighth embodiment, which is the method of any one of the first through the seventh embodiments, wherein the first reactant mixture is characterized by a CH₄/O₂ molar ratio of from about 2:1 to about 40:1.

A ninth embodiment, which is the method of any one of the first through the eighth embodiments, wherein the first reactant mixture further comprises hydrogen (H₂).

A tenth embodiment, which is the method of any one of the first through the ninth embodiments, wherein the second reactant mixture is characterized by a C₂H₆/CH₄ molar ratio of from 0.01 to about 0.5:1.

An eleventh embodiment, which is the method of any one of the first through the tenth embodiments, wherein the second H₂/CO molar ratio is controlled by varying a C₂H₆/CH₄ molar ratio of the second reactant mixture.

A twelfth embodiment, which is the method of any one of the first through the eleventh embodiments, wherein the second reaction zone is characterized by a gas hourly space velocity of from about 30 h⁻¹ to about 20,000 h⁻¹.

A thirteenth embodiment, which is the method of the third embodiment, wherein the first reactor comprises a non-catalytic OCM reactor, and wherein the first reaction zone and/or the second reaction zone is characterized by a gas hourly space velocity of from about 30 h⁻¹ to about 20,000 h⁻¹.

A fourteenth embodiment, which is the method of the third embodiment, wherein the first reactor comprises a catalytic OCM reactor comprising an OCM catalyst, and wherein the first reaction zone is characterized by a gas hourly space velocity of from about 3,600 h⁻¹ to about 36,000 h⁻¹.

A fifteenth embodiment, which is the method of the first embodiment, wherein the first reaction zone and the second reaction zone exclude a catalyst.

A sixteenth embodiment, which is the method of the second embodiment, wherein the common reactor comprises an isothermal reactor, a fluidized sand bath reactor, an autothermal reactor, an adiabatic reactor, a tubular reactor, a cooled tubular reactor, a continuous flow reactor, a reactor lined with an inert refractory material, a glass lined reactor, a ceramic lined reactor, or combinations thereof.

A seventeenth embodiment, which is the method of the sixteenth embodiment, wherein the inert refractory material comprises silica, alumina, silicon carbide, boron nitride, titanium oxide, mullite, mixtures of oxides, or combinations thereof.

An eighteenth embodiment, which is the method of the first embodiment, wherein the catalyst for the third reaction zone comprises Cu, Cu/ZnO, Cu/ThO₂, Cu/Zn/Al₂O₃, Cu/ZnO/Al₂O₃, Cu/Zr, or combinations thereof.

A nineteenth embodiment, which is the method of the fifth embodiment, wherein the OCM catalyst comprises basic oxides; mixtures of basic oxides; redox elements; redox elements with basic properties; mixtures of redox elements with basic properties; mixtures of redox elements with basic properties promoted with alkali and/or alkaline earth metals; rare earth metal oxides; mixtures of rare earth metal oxides; mixtures of rare earth metal oxides promoted by alkali and/or alkaline earth metals; manganese; manganese compounds; lanthanum; lanthanum compounds; sodium; sodium compounds; cesium; cesium compounds; calcium; calcium compounds; or combinations thereof.

A twentieth embodiment, which is the method of the fifth embodiment, wherein the OCM catalyst comprises CaO, MgO, BaO, CaO—MgO, CaO—BaO, Li/MgO, MnO₂, W₂O₃, SnO₂, MnO₂—W₂O₃, MnO₂—W₂O₃—Na₂O, MnO₂—W₂O₃—Li₂O, La₂O₃, SrO/La₂O₃, CeO₂, Ce₂O₃, La/MgO, La₂O₃—CeO₂, La₂O₃—CeO₂—Na₂O, La₂O₃—CeO₂—CaO, Na—Mn—La₂O₃/Al₂O₃, Na—Mn—O/SiO₂, Na₂WO₄—Mn/SiO₂, Na₂WO₄—Mn—O/SiO₂, or combinations thereof.

A twenty-first embodiment, which is the method of the first embodiment, wherein at least a portion of the water is removed from the second product mixture to yield a dehydrated second product mixture.

A twenty-second embodiment, which is the method the twenty-first embodiment, wherein at least a portion of the dehydrated second product mixture is contacted with a purified natural gas stream to yield a methane-rich product mixture, wherein an amount of methane in the methane-rich product mixture is greater than an amount of methane in the dehydrated second product mixture.

A twenty-third embodiment, which is the method of the twenty-second embodiment, wherein the purified natural gas stream is produced by desulphurization of a natural gas stream.

A twenty-fourth embodiment, which is the method of any one of the twenty-second and the twenty-third embodiments, wherein at least a portion of CO₂ is removed from the methane-rich product mixture to yield a purified methane-rich product mixture.

A twenty-fifth embodiment, which is the method of the twenty-fourth embodiment, wherein the methanol production feed stream and a C₂₊ hydrocarbons stream are recovered from at least a portion of the purified methane-rich product mixture by cryogenic distillation.

A twenty-sixth embodiment, which is the method of the twenty-fifth embodiment further comprising recovering a portion of H₂ from the methanol production feed stream by pressure swing adsorption to yield a recovered H₂ stream.

A twenty-seventh embodiment, which is the method of the first embodiment, wherein at least a portion of the methanol production feed stream is further contacted with additional hydrogen prior to (f) introducing at least a portion of the methanol production feed stream to the third reaction zone.

A twenty-eighth embodiment, which is the method of the twenty-fifth embodiment, wherein a first C₂ hydrocarbons stream and a C₃₊ hydrocarbons stream are recovered from at least a portion of C₂₊ hydrocarbons stream by cryogenic distillation, wherein the first C₂ hydrocarbons stream comprises ethylene (C₂H₄), ethane (C₂H₆), and acetylene (C₂H₂), and wherein the C₃₊ hydrocarbons stream comprises C₃ hydrocarbons and C₄ hydrocarbons.

A twenty-ninth embodiment, which is the method of the twenty-eighth embodiment, wherein at least a portion of the C₂H₂ in the first C₂ hydrocarbons stream is contacted with H₂ to yield a second C₂ hydrocarbons stream comprising ethylene, and ethane.

A thirtieth embodiment, which is the method of the twenty-ninth embodiment, wherein at least a portion of the recovered H₂ stream is recycled to the H₂ used for selective hydrogenation of C₂H₂.

A thirty-first embodiment, which is the method of any one of the twenty-eighth and the twenty-ninth embodiments, wherein an ethylene stream and the ethane stream are recovered from at least a portion of the second C₂ hydrocarbons stream by cryogenic distillation.

A thirty-second embodiment, which is the method of the first embodiment, wherein the ethane stream is further contacted with additional ethane prior to (c) introducing at least a portion of the first product mixture and the ethane stream to the second reaction zone.

A thirty-third embodiment, which is the method of the twenty-eighth embodiment, wherein a C₃ hydrocarbons stream and a C₄ hydrocarbons stream are recovered from at least a portion of the C₃₊ hydrocarbons stream, wherein the C₃ hydrocarbons comprise propylene (C₃H₆), and propane (C₃H₈).

A thirty-fourth embodiment, which is the method of any one of the first through the thirty-third embodiments further comprising introducing steam to the second reaction zone.

A thirty-fifth embodiment, which is the method of any one of the first through the thirty-fourth embodiments, wherein a per pass hydrocarbon conversion is from about 10% to about 50%.

A thirty-sixth embodiment, which is the method of any one of the first through the thirty-fifth embodiments, wherein an overall hydrocarbon yield is from about 50% to about 95%.

A thirty-seventh embodiment, which is the method of any one of the first through the thirty-sixth embodiments, wherein a selectivity to OCM primary products is from about 50% to about 95%.

A thirty-eighth embodiment, which is the method of the first embodiment, wherein a first selectivity to C₂₊ hydrocarbons is from about 25% to about 90%.

A thirty-ninth embodiment, which is the method of the first embodiment, wherein a first selectivity to C₂ hydrocarbons is from about 23% to about 85%.

A fortieth embodiment, which is the method of the first embodiment, wherein a first selectivity to ethylene is from about 22% to about 70%.

A forty-first embodiment, which is the method of the first embodiment, wherein a second selectivity to ethylene is from about 60% to about 98%.

A forty-second embodiment, which is the method of any one of the first through the forty-first embodiments, wherein the first H₂/CO molar ratio is from about 0.3:1 to about 2:1.

A forty-third embodiment, which is the method of any one of the first through the forty-second embodiments, wherein the second H₂/CO molar ratio is from about 0.5:1 to about 2.5:1.

A forty-fourth embodiment, which is the method of any one of the first through the forty-third embodiments, wherein equal to or greater than about 5 mol % of methane in the first reactant mixture is converted overall to useful recovered products, wherein the useful recovered products comprise C₂ hydrocarbons, C₃ hydrocarbons, C₄s, CH₃OH, or combinations thereof.

A forty-fifth embodiment, which is the method of any one of the first through the forty-fourth embodiments, wherein equal to or greater than about 5 mol % of methane in the first reactant mixture is converted overall to methanol.

A forty-sixth embodiment, which is the method of any one of the first through the forty-fifth embodiments, wherein the second product mixture comprises less than about 15 mol % carbon dioxide (CO₂).

A forty-seventh embodiment, which is the method of any one of the first through the forty-sixth embodiments further comprising minimizing deep oxidation of methane to carbon dioxide (CO₂).

A forty-eighth embodiment, which is a method for producing ethylene and methanol comprising (a) introducing a first reactant mixture to a first reaction zone, wherein the first reactant mixture comprises methane (CH₄) and oxygen (O₂), wherein the first reaction zone is characterized by a first reaction zone temperature of from about 800° C. to about 1,000° C., wherein the first reaction zone is characterized by a residence time of from about 250 milliseconds to about 750 milliseconds, and wherein the first reaction zone excludes a catalyst; (b) allowing at least a portion of the first reactant mixture in the first reaction zone to react via an oxidative coupling of CH₄ reaction to form a first product mixture, wherein the first product mixture comprises C₂₊ hydrocarbons, hydrogen (H₂), carbon monoxide (CO), water, CO₂, and unreacted methane, wherein the first product mixture is characterized by a first hydrogen (H₂) to carbon monoxide (CO) (H₂/CO) molar ratio of from about 0.5:1 to about 2.0:1, wherein the C₂₊ hydrocarbons comprise C₂ hydrocarbons and C₃₊ hydrocarbons, and wherein the C₂ hydrocarbons comprise ethane and ethylene; (c) introducing a second reactant mixture comprising at least a portion of the first product mixture and an ethane stream to a second reaction zone, wherein the second reactant mixture is characterized by a C₂H₆/CH₄ molar ratio of from about 0.01:1 to about 0.5:1, wherein the second reaction zone is characterized by a second reaction zone temperature of from about 800° C. to about 1,000° C., wherein the second reaction zone is characterized by a residence time of from about 200 milliseconds to about 800 milliseconds, and wherein at least a portion of ethane of the second reactant mixture undergoes a cracking reaction to produce ethylene; (d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio of from about 0.8:1 to about 2.5:1, and wherein the second H₂/CO molar ratio is greater than the first H₂/CO molar ratio; (e) recovering a methanol production feed stream from at least a portion of the second product mixture, wherein the methanol production feed stream comprises methane, H₂ and CO; and (f) introducing at least a portion of the methanol production feed stream to a third reaction zone comprising a Cu/Zn/Al₂O₃ catalyst to produce a methanol stream and a methane-rich stream, wherein at least a portion of the methane-rich stream is recycled to the first reaction zone.

A forty-ninth embodiment, which is the method of the forty-eighth embodiment, wherein a common reactor comprises both the first reaction zone and the second reaction zone.

A fiftieth embodiment, which is a method for producing olefins and methanol comprising (a) introducing a first reactant mixture to a first reaction zone, wherein the first reactant mixture comprises methane (CH₄) and oxygen (O₂), and wherein the first reaction zone is characterized by a first reaction zone temperature of from about 700° C. to about 1,100° C.; (b) allowing at least a portion of the first reactant mixture to react via an oxidative coupling of CH₄ reaction to form a first product mixture, wherein the first product mixture comprises C₂₊ hydrocarbons, hydrogen (H₂), carbon monoxide (CO), water, CO₂, and unreacted methane, wherein the first product mixture is characterized by a first hydrogen (H₂) to carbon monoxide (CO) (H₂/CO) molar ratio, wherein the C₂₊ hydrocarbons comprise C₂ hydrocarbons and C₃₊ hydrocarbons, and wherein the C₂ hydrocarbons comprise ethane (C₂H₆) and ethylene (C₂H₄); (c) introducing a second reactant mixture comprising at least a portion of the first product mixture and an ethane stream to a second reaction zone, wherein the second reaction zone is characterized by a second reaction zone temperature of from about 750° C. to about 1,000° C., and wherein at least a portion of ethane of the second reactant mixture undergoes a cracking reaction to produce ethylene; (d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio, and wherein the second H₂/CO molar ratio is greater than the first H₂/CO molar ratio; (e) recovering ethylene from at least a portion of the second product mixture; and (f) recovering at least a portion of the H₂ and at least a portion of the CO from the second product mixture to yield a recovered synthesis gas.

A fifty-first embodiment, which is the method of the fiftieth embodiment, wherein the recovered synthesis gas is characterized by a H₂/CO molar ratio of about 2:1, and wherein at least a portion of the recovered synthesis gas is used for methanol production.

A fifty-second embodiment, which is the method of the fiftieth embodiment, wherein the recovered synthesis gas is characterized by a H₂/CO molar ratio of about 1:1, and wherein at least a portion of the recovered synthesis gas is used for dimethyl ether production.

A fifty-third embodiment, which is the method of the fiftieth embodiment, wherein the recovered synthesis gas is characterized by a H₂/CO molar ratio of about 1:1, and wherein at least a portion of the recovered synthesis gas is used for oxo-synthesis of aliphatic aldehydes and/or alcohols.

A fifty-fourth embodiment, which is the method of the fiftieth embodiment, wherein at least a portion of the recovered synthesis gas is further converted to olefins.

A fifty-fifth embodiment, which is the method of the fiftieth embodiment, wherein at least a portion of the recovered synthesis gas is further converted to liquid hydrocarbons by a Fischer-Tropsch process.

A fifty-sixth embodiment, which is the method of the fiftieth embodiment, wherein at least a portion of the recovered synthesis gas is further used as fuel to generate power.

A fifty-seventh embodiment, which is the method of the fiftieth embodiment, wherein at least a portion of the recovered synthesis gas is further converted to methane via a methanation process.

A fifty-eighth embodiment, which is a method for producing olefins and methanol comprising (a) reacting, in the absence of a catalyst, methane (CH₄) and oxygen (O₂) via an oxidative coupling reaction in a first reaction zone to form a first product mixture, wherein the first product mixture comprises ethane, ethylene, carbon monoxide (CO), hydrogen (H₂), water, carbon dioxide (CO₂), and unreacted methane; (b) introducing at least a portion of the first product mixture to a second reaction zone, wherein at least a portion of the ethane undergoes a [steam] cracking reaction to produce ethylene; (c) recovering a second product mixture from the second reaction zone, wherein the second product mixture comprises ethane, ethylene, carbon monoxide (CO), hydrogen (H₂), water, CO₂, and unreacted methane; and (d) reacting at least a portion of the unreacted methane, CO, and H₂ from the second product mixture in a third reaction zone to form methanol.

A fifty-ninth embodiment, which is the method of the fifty-eighth embodiment, wherein a H₂/CO molar ratio of the first product mixture is from about 0.5:1 to about 2.0:1.

A sixtieth embodiment, which is the method of any one of the fifty-eighth and the fifty-ninth embodiments, wherein a H₂/CO molar ratio of the second product mixture is from about 0.6:1 to about 2.0:1.

A sixty-first embodiment, which is a method for producing olefins and methanol comprising (a) introducing a first reactant mixture to a first reaction zone, wherein the first reactant mixture comprises methane (CH₄) and oxygen (O₂), wherein the first reaction zone is characterized by a first reaction zone temperature of from about 800° C. to about 1,000° C., wherein the first reaction zone is characterized by a residence time in a catalyst bed of from about 20 milliseconds to about 50 milliseconds, and wherein the first reaction zone is catalyzed by an OCM catalyst comprising 2% MnO-5% Na₂WO₄/SiO₂; (b) allowing at least a portion of the first reactant mixture in the first reaction zone to react via an oxidative coupling of CH₄ reaction to form a first product mixture, wherein the first product mixture comprises C₂₊ hydrocarbons, hydrogen (H₂), carbon monoxide (CO), water, carbon dioxide (CO₂), and unreacted methane, wherein the first product mixture is characterized by a first H₂/CO molar ratio of from about 0.5:1 to about 1:1, wherein the C₂₊ hydrocarbons comprise C₂ hydrocarbons and C₃₊ hydrocarbons, and wherein the C₂ hydrocarbons comprise ethane and ethylene; (c) introducing a second reactant mixture comprising at least a portion of the first product mixture and an ethane stream to a second reaction zone, wherein the second reactant mixture is characterized by a C₂H₆/CH₄ molar ratio of from about 0.01:1 to about 0.5:1, wherein the second reaction zone is characterized by a second reaction zone temperature of from about 800° C. to about 1,000° C., wherein the second reaction zone is characterized by a residence time of from about 200 milliseconds to about 800 milliseconds, and wherein at least a portion of ethane of the second reactant mixture undergoes a cracking reaction to produce ethylene; (d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio of from about 0.8:1 to about 2.5:1, and wherein the second H₂/CO molar ratio is greater than the first H₂/CO molar ratio; (e) recovering a methanol production feed stream from at least a portion of the second product mixture, wherein the methanol production feed stream comprises methane, H₂ and CO; and (f) introducing at least a portion of the methanol production feed stream to a third reaction zone comprising a Cu/Zn/Al₂O₃ catalyst to produce a methanol stream and a methane-rich stream, wherein at least a portion of the methane-rich stream is recycled to the first reaction zone.

A sixty-second embodiment, which is the method of the sixty-first embodiment, wherein a common reactor comprises both the first reaction zone and the second reaction zone.

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

What is claimed is:

1. A method for producing olefins and methanol comprising:
(a) introducing a first reactant mixture to a first reaction zone, wherein the first reactant mixture comprises methane (CH₄) and oxygen (O₂), and wherein the first reaction zone is characterized by a first reaction zone temperature of from about 700° C. to about 1,100° C.;
(b) allowing at least a portion of the first reactant mixture to react via an oxidative coupling of CH₄ (OCM) reaction to form a first product mixture, wherein the first product mixture comprises C₂₊ hydrocarbons, hydrogen (H₂), carbon monoxide (CO), water, CO₂, and unreacted methane, wherein the first product mixture is characterized by a first hydrogen (H₂) to carbon monoxide (CO) (H₂/CO) molar ratio, wherein the C₂₊ hydrocarbons comprise C₂ hydrocarbons and C₃₊ hydrocarbons, and wherein the C₂ hydrocarbons comprise ethane (C₂H₆) and ethylene (C₂H₄);
(c) introducing a second reactant mixture comprising at least a portion of the first product mixture and an ethane stream to a second reaction zone, wherein the second reaction zone is characterized by a second reaction zone temperature of from about 750° C. to about 1,000° C., and wherein at least a portion of ethane of the second reactant mixture undergoes a cracking reaction to produce ethylene;
(d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio, and wherein the second H₂/CO molar ratio is greater than the first H₂/CO molar ratio;
(e) recovering a methanol production feed stream from at least a portion of the second product mixture, wherein the methanol production feed stream comprises methane, H₂ and CO; and
(f) introducing at least a portion of the methanol production feed stream to a third reaction zone comprising a catalyst to produce a methanol stream and a methane-rich stream, wherein at least a portion of the methane-rich stream is recycled to the first reaction zone.

2. The method of claim 1, wherein a common reactor comprises both the first reaction zone and the second reaction zone.

3. The method of claim 1, wherein a first reactor comprises the first reaction zone, and wherein a second reactor comprises the second reaction zone, wherein the first reactor comprises a non-catalytic OCM reactor, and wherein the first reaction zone is characterized by a residence time of from about 100 milliseconds to about 30 seconds.

4. The method of claim 1, wherein a first reactor comprises the first reaction zone, and wherein a second reactor comprises the second reaction zone, wherein the first reactor comprises a catalytic OCM reactor, and wherein the first reaction zone is characterized by a residence time in a catalyst bed of from about 10 milliseconds to about 200 milliseconds.

5. The method of claim 1, wherein the second reaction zone is characterized by a residence time of from about 100 milliseconds to about 2 seconds.

6. A method for producing ethylene and methanol comprising:
(a) introducing a first reactant mixture to a first reaction zone, wherein the first reactant mixture comprises methane (CH₄) and oxygen (O₂), wherein the first reaction zone is characterized by a first reaction zone temperature of from about 800° C. to about 1,000° C., wherein the first reaction zone is characterized by a residence time of from about 250 milliseconds to about 750 milliseconds, and wherein the first reaction zone excludes a catalyst;
(b) allowing at least a portion of the first reactant mixture in the first reaction zone to react via an oxidative coupling of CH₄ reaction to form a first product mixture, wherein the first product mixture comprises C₂₊ hydrocarbons, hydrogen (H₂), carbon monoxide (CO), water, CO₂, and unreacted methane, wherein the first product mixture is characterized by a first hydrogen (H₂) to carbon monoxide (CO) (H₂/CO) molar ratio of from about 0.5:1 to about 2.0:1, wherein the C₂₊ hydrocarbons comprise C₂ hydrocarbons and C₃₊ hydrocarbons, and wherein the C₂ hydrocarbons comprise ethane and ethylene;
(c) introducing a second reactant mixture comprising at least a portion of the first product mixture and an ethane stream to a second reaction zone, wherein the second reactant mixture is characterized by a C₂H₆/CH₄ molar ratio of from about 0.01:1 to about 0.5:1, wherein the second reaction zone is characterized by a second reaction zone temperature of from about 800° C. to about 1,000° C., wherein the second reaction zone is characterized by a residence time of from about 200 milliseconds to about 800 milliseconds, and wherein at least a portion of ethane of the second reactant mixture undergoes a cracking reaction to produce ethylene;
(d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio of from about 0.8:1 to about 2.5:1, and wherein the second H₂/CO molar ratio is greater than the first H₂/CO molar ratio;
(e) recovering a methanol production feed stream from at least a portion of the second product mixture, wherein the methanol production feed stream comprises methane, H₂ and CO; and
(f) introducing at least a portion of the methanol production feed stream to a third reaction zone comprising a Cu/Zn/Al₂O₃ catalyst to produce a methanol stream and a methane-rich stream, wherein at least a portion of the methane-rich stream is recycled to the first reaction zone.

7. The method of claim 6, wherein a common reactor comprises both the first reaction zone and the second reaction zone.

8. A method for producing olefins and methanol comprising:
(a) introducing a first reactant mixture to a first reaction zone, wherein the first reactant mixture comprises methane (CH₄) and oxygen (O₂), and wherein the first reaction zone is characterized by a first reaction zone temperature of from about 700° C. to about 1,100° C.;
(b) allowing at least a portion of the first reactant mixture to react via an oxidative coupling of CH₄ reaction to form a first product mixture, wherein the first product mixture comprises C₂₊ hydrocarbons, hydrogen (H₂), carbon monoxide (CO), water, CO₂, and unreacted methane, wherein the first product mixture is characterized by a first hydrogen (H₂) to carbon monoxide (CO) (H₂/CO) molar ratio, wherein the C₂₊ hydrocarbons comprise C₂ hydrocarbons and C₃₊ hydrocarbons, and wherein the C₂ hydrocarbons comprise ethane (C₂H₆) and ethylene (C₂H₄);
(c) introducing a second reactant mixture comprising at least a portion of the first product mixture and an ethane stream to a second reaction zone, wherein the second reaction zone is characterized by a second reaction zone temperature of from about 750° C. to about 1,000° C., and wherein at least a portion of ethane of the second reactant mixture undergoes a cracking reaction to produce ethylene;
(d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio, and wherein the second H₂/CO molar ratio is greater than the first H₂/CO molar ratio;
(e) recovering ethylene from at least a portion of the second product mixture; and
(f) recovering at least a portion of the H₂ and at least a portion of the CO from the second product mixture to yield a recovered synthesis gas.

9. The method of claim 8, wherein the recovered synthesis gas is characterized by a H₂/CO molar ratio of about 2:1, and wherein at least a portion of the recovered synthesis gas is used for methanol production.

10. The method of claim 8, wherein the recovered synthesis gas is characterized by a H₂/CO molar ratio of about 1:1, and wherein at least a portion of the recovered synthesis gas is used for dimethyl ether production.

11. The method of claim 8, wherein the recovered synthesis gas is characterized by a H₂/CO molar ratio of about 1:1, and wherein at least a portion of the recovered synthesis gas is used for oxo-synthesis of aliphatic aldehydes and/or alcohols.

12. The method of claim 8, wherein at least a portion of the recovered synthesis gas is further converted to olefins.

13. The method of claim 8, wherein at least a portion of the recovered synthesis gas is further converted to liquid hydrocarbons by a Fischer-Tropsch process.

14. The method of claim 8, wherein at least a portion of the recovered synthesis gas is further used as fuel to generate power.

15. The method of claim 8, wherein at least a portion of the recovered synthesis gas is further converted to methane via a methanation process.

16. A method for producing olefins and methanol comprising:
(a) reacting, in the absence of a catalyst, methane (CH₄) and oxygen (O₂) via an oxidative coupling reaction in a first reaction zone to form a first product mixture, wherein the first product mixture comprises ethane, ethylene, carbon monoxide (CO), hydrogen (H₂), water, carbon dioxide (CO₂), and unreacted methane;
(b) introducing at least a portion of the first product mixture to a second reaction zone, wherein at least a portion of the ethane undergoes a steam cracking reaction to produce ethylene;
(c) recovering a second product mixture from the second reaction zone, wherein the second product mixture comprises ethane, ethylene, carbon monoxide (CO), hydrogen (H₂), water, CO₂, and unreacted methane; and
(d) reacting at least a portion of the unreacted methane, CO, and H₂ from the second product mixture in a third reaction zone to form methanol.

17. The method of claim 16, wherein a H₂/CO molar ratio of the first product mixture is from about 0.5:1 to about 2.0:1.

18. The method of claim 16, wherein a H₂/CO molar ratio of the second product mixture is from about 0.6:1 to about 2.0:1.

19. A method for producing olefins and methanol comprising:
(a) introducing a first reactant mixture to a first reaction zone, wherein the first reactant mixture comprises methane (CH₄) and oxygen (O₂), wherein the first reaction zone is characterized by a first reaction zone temperature of from about 800° C. to about 1,000° C., wherein the first reaction zone is characterized by a residence time in a catalyst bed of from about 20 milliseconds to about 50 milliseconds, and wherein the first reaction zone is catalyzed by an OCM catalyst comprising 2% MnO-5% Na₂WO₄/SiO₂;
(b) allowing at least a portion of the first reactant mixture in the first reaction zone to react via an oxidative coupling of CH₄ reaction to form a first product mixture, wherein the first product mixture comprises C₂₊ hydrocarbons, hydrogen (H₂), carbon monoxide (CO), water, carbon dioxide (CO₂), and unreacted methane, wherein the first product mixture is characterized by a first H₂/CO molar ratio of from about 0.5:1 to about 1:1, wherein the C₂₊ hydrocarbons comprise C₂ hydrocarbons and C₃₊ hydrocarbons, and wherein the C₂ hydrocarbons comprise ethane and ethylene;
(c) introducing a second reactant mixture comprising at least a portion of the first product mixture and an ethane stream to a second reaction zone, wherein the second reactant mixture is characterized by a C₂H₆/CH₄ molar ratio of from about 0.01:1 to about 0.5:1, wherein the second reaction zone is characterized by a second reaction zone temperature of from about 800° C. to about 1,000° C., wherein the second reaction zone is characterized by a residence time of from about 200 milliseconds to about 800 milliseconds, and wherein at least a portion of ethane of the second reactant mixture undergoes a cracking reaction to produce ethylene;
(d) recovering a second product mixture from the second reaction zone, wherein the second product mixture comprises C₂₊ hydrocarbons, H₂, CO, water, CO₂, and unreacted methane, wherein the second product mixture is characterized by a second H₂/CO molar ratio of from about 0.8:1 to about 2.5:1, and wherein the second H₂/CO molar ratio is greater than the first H₂/CO molar ratio;
(e) recovering a methanol production feed stream from at least a portion of the second product mixture, wherein the methanol production feed stream comprises methane, H₂ and CO; and
(f) introducing at least a portion of the methanol production feed stream to a third reaction zone comprising a Cu/Zn/Al₂O₃ catalyst to produce a methanol stream and a methane-rich stream, wherein at least a portion of the methane-rich stream is recycled to the first reaction zone.

20. The method of claim 19, wherein a common reactor comprises both the first reaction zone and the second reaction zone.
